(19) United States
(12) Patent Application Publication
(10) Pub. No.: US 2007/0212360 A1
(43) Pub. Date: Sep. 13, 2007

(54) Modulation of mitochondrial oxygen consumption for therapeutic purposes

(76) Inventors: Nicholas C. Denko, Menlo Park, CA (US); Robert A. Cairns, Palo Alto, CA (US); Ioanna Papandreou, Palo Alto, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 11/654,796

(22) Filed: Jan. 17, 2007

Related U.S. Application Data
(60) Provisional application No. 60/759,680, filed on Jan. 17, 2006.

(51) Int. Cl. A61K 39/395; A61K 31/404

(52) U.S. Cl. 424/155.1; 424/178.1; 514/418; 514/410

(57) ABSTRACT

The HIF-1 transcription factor drives gene expression changes in hypoxia. While HIF-1 stimulates glycolysis, it also actively represses mitochondrial function and oxygen consumption by inducing pyruvate dehydrogenase kinase 1 (PDK1). PDK1 phosphorylates and inhibits pyruvate dehydrogenase from converting pyruvate to acetyl-CoA to fuel the mitochondrial TCA cycle. This causes a drop in mitochondrial oxygen utilization and results in a relative increase in intracellular oxygen tension.

### INTRODUCTION

Tissue hypoxia results when supply of oxygen from the bloodstream does not meet demand from the cells in the tissue. Such a supply-demand mismatch can occur in physiologic conditions such as the exercising muscle, or in the pathologic condition such as the ischemic heart, or in the tumor microenvironment. In either the physiologic circumstance, or pathologic conditions, there is a molecular response from the cell in which a program of gene expression changes is initiated by the hypoxia-inducible factor-1 (HIF-1) transcription factor. This program of gene expression changes is thought to help the cells adapt to the stressful environment. For example, HIF-1 dependent expression of erythropoietin and angiogenic compounds results in increased blood vessel formation for delivery of a richer supply of oxygenated blood to the hypoxic tissue. Additionally, HIF-1 induction of glycolytic enzymes allows for production of energy when the mitochondria are starved of oxygen as a substrate for oxidative phosphorylation. We now find that this metabolic adaptation is more complex, with HIF-1 not only regulating the supply of oxygen from the bloodstream, but also actively regulating the oxygen demand of the tissue by reducing the activity of the major cellular consumer of oxygen, the mitochondria.

Perhaps the best studied example of chronic hypoxia is the hypoxia associated with the tumor microenvironment. The tumor suffers from poor oxygen supply through a chaotic jumble of blood vessels that are unable to adequately perfuse the tumor cells. The oxygen tension within the tumor is also a function of the demand within the tissue, with oxygen consumption influencing the extent of tumor hypoxia. The net result is that a large fraction of the tumor cells are hypoxic. Oxygen tensions within the tumor range from near normal at the capillary wall, to near zero in the perinecrotic regions. This perfusion-limited hypoxia is a potent microenvironmental stress during tumor evolution and an important variable capable of predicting for poor patient outcome.

The HIF-1 transcription factor was first identified based on its ability to activate the erythropoetin gene in response to hypoxia. Since then, it is has been shown to be activated by hypoxia in many cells and tissues, where it can induce hypoxia-responsive target genes such as VEGF and Glut1. The connection between HIF-regulation and human cancer was directly linked when it was discovered that the VHL tumor suppressor gene was part of the molecular complex responsible for the oxic degradation of HIF-1α. In normoxia, a family of prolyl hydroxylase enzymes uses molecular oxygen as a substrate and modifies HIF-1α and HIF2α by hydroxylation of prolines 564 and 402. VHL then recognizes the modified HIF-α proteins, acts as an E3-type of ubiquitin ligase, and along with elongins B and C is responsible for the polyubiquitination of HIF-αs and their proteosomal degradation. Mutations in VHL lead to constitutive HIF-1 gene expression, and predispose humans to cancer. The ability to recognize modified HIF-αs is at least partly responsible for VHL activity as a tumor suppressor, as introduction of non-degradable HIF-2α is capable of overcoming the growth-inhibitory activity of wild-type VHL in renal cancer cells.

Mitochondrial function can be regulated by PDK1 expression. Mitochondrial oxidative phosphorylation (OXPHOS) is regulated by several mechanisms, including substrate availability. The major substrates for OXPHOS are oxygen which is the terminal electron acceptor, and pyruvate, which is the primary carbon source. Pyruvate is the end product of glycolysis, and is converted to acetylCoA through the activity of the pyruvate dehydrogenase complex of enzymes. The Acetyl-CoA then directly enters the TCA cycle at citrate synthase where it is combined with oxaloacetate to generate citrate. In metazoans, the conversion of pyruvate to Acetyl-CoA is irreversible, and therefore represents a critical regulatory point in cellular energy metabolism. Pyruvate dehydrogenase is regulated by three known mechanisms: it is inhibited by Acetyl-CoA and NADH, it is stimulated by reduced energy in the cell, and it is inhibited by regulatory phosphorylation of its E1 subunit by Pyruvate Dehydrogenase Kinase (PDK). There are four members of the PDK family in vertebrates, each with specific tissue distributions. PDK expression has been observed in human tumor biopsies, and it has been reported that PDK3 is hypoxia-inducible in some cell types.

### SUMMARY OF THE INVENTION

The present invention provides methods that utilize regulation of mitochondrial oxidative phosphorylation to increase susceptibility to hypoxic cytotoxins. In particular, such methods find use in the treatment of cancer. In one embodiment of the invention, a hypoxic cytotoxin is administered in conjunction with an inhibitor of HIF-1. In another embodiment of the invention, a hypoxic cytotoxin is administered in conjunction with an inhibitor of PDK.

Tumor cells are contacted with an agent that modulates mitochondrial oxidative phosphorylation and a hypoxic cytotoxin, either locally or systemically. The combination provides for a synergistic effect, with comparable or improved therapeutic effects, while lowering adverse side effects.

In one embodiment, the invention provides pharmaceutical formulations comprising an effective dose of inhibitor of the invention, an effective dose of a hypoxic cytotoxin, and a pharmaceutically acceptable carrier.

These and other aspects and embodiments of the invention and methods for making and using the invention are described in more detail in the description of the drawings and the invention, the examples, the claims, and the drawings that follow.

### BRIEF DESCRIPTION OF THE DRAWINGS

FIG. 1. Hypoxia inhibits mitochondrial oxygen consumption. A: Oxygen concentration curves generated using a Clark electrode for fibroblast suspensions from cultures exposed to normoxia or hypoxia for 24 h. Arrows indicate addition of the mitochondrial uncoupler CCCP and cytochrome poison cyanide. The slope of the curve is a measure of the rate of oxygen consumption. B: Average oxygen consumption rates in normal human fibroblasts and immortalized murine fibroblasts cultured in normoxia or 0.5% O2 for 24 h. C: Fraction of total oxygen consumption due to mitochondrial versus non-mitochondrial consumption in wild-type murine fibroblasts. Mitochondrial consumption was determined using 1 ug/ml of the cell permeable complex 3 inhibitor Antimycin A. D: Change in oxygen consumption in RKO cells after exposure to the indicated time in 0.5% O2, and time to recovery in normoxia following 24 h hypoxia. For all graphs, the error bars represent the standard error of the mean.

FIG. 2. Hypoxic reduction in mitochondrial oxygen consumption is HIF-1 dependent. A: Average oxygen consumption rates in vhl-deficient RCC4 cells, RCC4/VHL cells and RCC4/VHLY98H cells after exposure to 24 h of normoxia or 0.5% O2. B: Average oxygen consumption rates in HIF wt and HIF ko murine fibroblasts after exposure to normoxia or 0.5% O2 for 24 h. C: Average oxygen consumption rates in parental RKO cells and ShRNAHIF-1α RKO cells after exposure to 24 h of normoxia or 0.5% O2.

FIG. 3. Mitochondria are not grossly altered by hypoxia or HIF activity. A: Fluorescent microscopy of RCC4 and RCC4/VHL cells under normoxic conditions shows no morphologic difference in mitochondria. In the top panels, cells were transiently transfected with mito-DsRed to label mitochondria and visualized with 594 nm excitation. In the bottom panels, immuno-fluorescence was used to visualize the endogenous mitochondrial protein cytochrome c and detected with anti mouse Alexa 488 secondary antibody. B: Mitochondrial membrane potential is not altered in RCC4, RCC4/VHL, RCC4/VHLY98H, HIF wt MEFs, and HIF ko MEFs after exposure to normoxia or 0.5% O2 for 24 h. Rhodamine 123 staining and flow cytometry was used to measure the mean fluorescence intensity, which is expressed relative to parental cells under normoxic conditions. Error bars represent the standard error of the mean. C: Western blot of lysates from the indicated cells exposed to normoxia or 0.5% O2 for 24 h were probed for mitochondrial HSP60, a constitutive mitochondrial protein, as a measure of total mitochondrial mass. Blots were reprobed for tubulin as a loading control.

FIG. 4. PDK1 protein is upregulated by hypoxia in a HIF dependant manner. A: Western blots of RCC4, RCC4/VHL, and RCC4/VHLY98H lysates after exposure to normoxia or 0.5% O2 for 24 h. Blots were probed for HIF 1α, PDK1, the HIF target gene BNip3, and tubulin as a loading control. B: Western blots of HIFwt and HIFko MEF lysates after exposure to the indicated oxygen concentrations for 24 h. Blots were probed for PDK1, the HIF target gene BNip3, and tubulin as a loading control. C: Western blots of ShRNABNip3RKO and ShRNAHIF-1αRKO lysates after exposure to normoxia or 0.5% O2 for 24 h. Blots were probed for HIF 1α, to demonstrate specific inhibition in the ShHIF 1α cell line, PDK1, the HIF target gene BNip3L, and tubulin as a loading control. Note that BNip3 is not expressed in RKO cells. D: Analysis of the PDK1 promoter for functional HREs. Segments of DNA taken from the indicated 5′ flanking region were fused to the firefly luciferase gene and transiently transfected into the wild-type and the HIF1α knockout cells as indicated. Cells were either co-transfected with 50 ng of empty vector or HIF1α (P402A/P564G) expression plasmid, or they were treated with 24 h 0.5% hypoxia as indicated. The relative luciferase activity was measured in a Beckton Dickenson Monolite 2000 and normalized for transfection efficiency using co-transfected renilla luciferase and empty pGL3 for a non-inducible construct. The baseline expression of the reporter genes is within 2 fold when comparing normoxic HIFwt to HIFko cells. Note the loss of HIF-dependent induction in the −36 to +30 fragment when the target HRE is mutated.

FIG. 5. PDK1 expression directly regulates cellular oxygen consumption rate. A: Western blot of RKO cell and ShRNAPDK1RKO cell lysates after exposure to 24 h of normoxia or 0.5% O2. Blots were probed for HIF 1α, PDK1, and tubulin as a loading control. B: Oxygen consumption rate in RKO and ShRNAPDK1 RKO cells after exposure to 24 h of normoxia or 0.5% O2. Error bars represent the standard error of the mean. C: Western blot of RKOiresGUS cell and RKOiresPDK1 cell lysates after exposure to 24 h of normoxia or 0.5% O2. Blots were probed for HIF 1α, PDK1, and tubulin as a loading control. D: Oxygen consumption rate in RKOiresGUS and RKOiresPDK1 cells after exposure to 24 h of normoxia or 0.5% O2. E Model describing the interconnected effects of HIF-1 target gene activation on hypoxic cell metabolism. Reduced oxygen conditions cause nuclear HIF-1 to coordinately induce the enzymes shown in boxes. HIF-1 activation results in increased glucose transporter expression to increase intracellular glucose flux, induction of glycolytic enzymes increases the conversion of glucose to pyruvate generating energy and NADH, induction of PDK1 decreases mitochondrial utilization of pyruvate and oxygen, and induction of LDH increases the removal of excess pyruvate as lactate and also regenerates NAD+ for increased glycolysis.

FIG. 6. HIF dependent decrease in oxygen consumption raises intracellular oxygen concentration, protects when oxygen is limiting, and decreases sensitivity to tirapazamine in vitro. A: Pimonidazole was used to determine the intracellular oxygen concentration of cells in culture. HIFwt and HIFko MEFs were grown at high density and exposed to 2% O2 or anoxia for 24 h in glass dishes. For the last 4 hours of treatment, cells were exposed to 60 μg/ml pimonidazole. Pimonidazole binding was quantitated by flow cytometry after binding of an FITC conjugated anti-pimo mAb. Results are representative of two independent experiments. B: HIF1α reduces oxygen consumption and protects cells when total oxygen is limited. HIFwt and HIFko cells were plated at high density and sealed in aluminum jigs at <0.02% oxygen. At the indicated times, cells were harvested and dead cells were quantitated by trypan blue exclusion. Note both cell lines are equally sensitive to anoxia-induced apoptosis, so the death of the HIF null cells indicates that the increased oxygen consumption removed any residual oxygen in the jig and resulted in anoxia-induced death. C. PDK1 is responsible for HIF-1's adaptive response when oxygen is limiting. A similar jig experiment was performed to measure survival in the parental RKO, the RKO ShRNAHIF1α and the RKOShPDK1 cells. Cell death by trypan blue uptake was measured 48 h after the jigs were sealed. D: HIF status alters sensitivity to TPZ in vitro. HIFwt and HIFko MEFs were grown at high density in glass dishes and exposed to 21%, 2%, and <0.01% O2 conditions for 18 hours in the presence of varying concentrations of Tirapazamine. After exposure, cells were harvested, and replated under normoxia to determine clonogenic viability. Survival is calculated relative to the plating efficiency of cells exposed to 0 μM TPZ for each oxygen concentration. Error bars represent the standard error of the mean. E: Cell density alters sensitivity to TPZ. HIFwt and HIFko MEFs were grown at varying cell densities in glass dishes and exposed to 2% O2 in the presence of 10 μM TPZ for 24 h. After the exposure, survival was determined as described in C.

FIG. 7. Inhibition of HIF1 and PDK1 increases oxygen consumption in vitro and in vivo. (a) Western blots of extracts from RKO and Su.86 cells exposed to normoxia (N) or hypoxia (H) (0.5% O2) for 24 h in the presence or absence of 2 ng/ml echinomycin probed for the indicated proteins. (b) Oxygen consumption rates of RKO and RKOShHIF1α cells (left) and Su.86 cells (right) after 24 h treatment with normoxia or hypoxia (0.5% O2) with or without 2 ng/ml echinomycin. Data are normalized to normoxic samples. (c) Relative oxygen consumption of RKO cells treated with hypoxia (0.5% O2) for 24 h in the presence of increasing concentrations of echinomycin. (d) Oxygen consumption rates of freshly explanted tumor tissue from RKO (n=16) and RKOShHIF1α (n=8) xenografts. (e) Oxygen consumption rates of RKO and RKOShHIF1 tumors from mice treated with 0.12 mg/kg echinomycin ip 24 h prior (n=8), or 50 mg/kg DCA ip 4 h prior (n=8). Data is normalized to PBS treated controls (n=8-16). (*) indicate a significant difference relative to control (p<0.05).

FIG. 8. Increasing oxygen consumption by inhibition of PDK activity increases tumor hypoxia. (a) Luciferase activity of wildtype and HIF1α knockdown RKO cells stably transfected with a HIF1 responsive luciferase reporter gene. Cells were exposed to 0.5% O2 for 24 hours in triplicate. Luminescence is normalized to normoxic HIF wildtype cells. (b) Luciferase activity of wildtype RKO HIF1 reporter cells exposed to 0.5% O2 for 24 hours in the presence of increasing concentrations of echinomycin. Data are normalized to the increase in signal observed in the absence of drug. (c) Bioluminescent imaging in vivo. Images show a representative animal bearing an RKO reporter tumor on the left flank and an RKOShHIF1α reporter tumor on the right flank as a function of time after ip injection of 50 mg/kg DCA. The pseudocolor overlay shows the intensity of bioluminescence. (d) Quantification of in vivo bioluminescence. The graph shows the change in signal intensity after DCA treatment for RKO parent and RKOShHIF1α tumors. The data represent the mean of three independent experiments, each comprising 5 RKO and 5 RKOShHIF1α reporter tumors.

FIG. 9. Increasing oxygen consumption by inhibition of HIF increases tumor hypoxia. (a-d) Pimonidazole staining of tumor sections from RKO (a,b) and RKOShHIF1α (c,d) tumors 24 h after treatment with PBS (a,c) or echinomycin (b,d). The tumor section is outlined in white, pimonidazole staining is shown in green, and the necrotic areas and cutting artifacts are shown in gray. (e) The mean hypoxic fraction of RKO and RKOShHIF1α tumors 24 h after treatment with PBS or echinomycin (n=4-5 tumors per group). Error bars represent the standard error of the mean. (*) indicates a significant difference (p<0.05).

FIG. 10. Pharmacologic inhibition of HIF1 or PDK1 enhances the response of tumor xenografts to the hypoxic cytotoxin tirapazamine. For all experiments, treatment was initiated when the mean tumor volume was 100-200 mm³. (a) RKO tumor bearing mice were treated with 0.12 mg/kg echinomycin ip followed by 30 mg/kg tirapazamine ip at 24 hours, followed by a rest day for 3 cycles. Single agents were given at the same dose, on the same schedule. (b) RKO tumor bearing mice were treated with 50 mg/kg DCA ip followed by 20 mg/kg tirapazamine ip at 4 hours daily for 14 days. Single agents were given at the same dose, on the same schedule. (c,d,e) RKO (c), RKOShHIF1α (d), or Su.86 (e) tumor bearing mice were treated with echinomycin plus tirapazamine as described above for 6 cycles (Ech+TPZ), or tirapazamine followed by echinomycin at 24 hours followed by a rest day for 6 cycles (TPZ+Ech). For all experiments, arrows indicate when treatment was stopped.

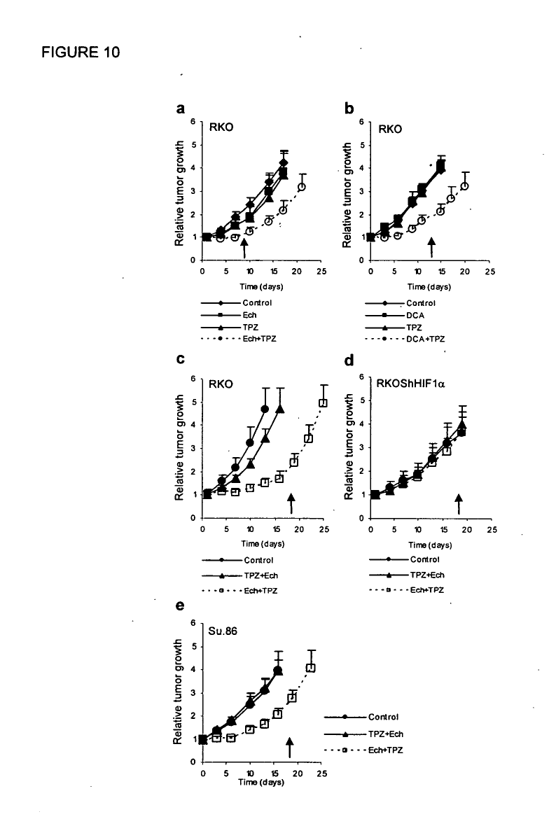

FIG. 11. Inhibition of HIF1 increases oxygen consumption in vitro. (a) Western blot of wildtype and HIF1α knockout MEFs exposed normoxia (N) or hypoxia (H) (0.5% O2) for 24 hours in the presence or absence of 2 ng/ml echinomycin. (b) In vitro oxygen consumption of wildtype and HIF1α knockout MEFs (b) after exposure to 24 hours of normoxia or hypoxia (0.5% O2) in the presence or absence of 2 ng/ml echinomycin. Data are normalized to normoxic values. (*) indicate a significant difference relative to control (p<0.05).

FIG. 12. Growth rates of RKO and RKOShHIF1α tumors implanted sc into 6-8 week old female nude mice.

FIG. 13. Oxygen consumption rates of Su.86 tumors from mice treated with 0.12 mg/kg echinomycin ip 24 h prior (n=4), or 50 mg/kg DCA ip 4 h prior (n=6). Data is normalized to PBS treated controls (n=6). (*) indicate a significant difference relative to control (p<0.05).

FIG. 14. Luciferase activity of hypoxia reporter tumors in response to tirapazamine. Mice bearing 500 mm³ RKO HRE-luciferase tumors were imaged using an IVIS100 bioluminescent imaging system, and the signal from each tumor was quantified. The graph shows the change in signal intensity over time after treatment with 60 mg/kg tirapazamine ip. The data represent the mean of 8 tumors.

### DETAILED DESCRIPTION OF THE EMBODIMENTS

The present invention provides methods that utilize regulation of mitochondrial oxidative phosphorylation to increase susceptibility to bioreductive drugs that target hypoxic cells (hypoxic cytotoxins). In particular, such methods find use in the treatment of cancer. In one embodiment of the invention, a hypoxic cytotoxin is administered in conjunction with an inhibitor of HIF-1. In another embodiment of the invention, a hypoxic cytotoxin is administered in conjunction with an inhibitor of PDK. Bioreductive drugs of interest include, without limitation, the benzotriazine di-N-oxide class of hypoxic cytotoxins, for example, tirapazamine. For a review of hypoxia activated drugs, see Denny (2004) Curr Med Chem Anticancer Agents. 4(5):395-9, herein specifically incorporated by reference for the teachings relating to such drugs.

For the treatment of cancer, the HIF-1 or PDK inhibitors act as a sensitizing agent, which enhance killing by hypoxic cytotoxins. For sensitization, the inhibitor may be administered separately or in a co-formulation with a bioreductive agent. Although the bioreductive agents may be active when administered alone, the concentrations required for a therapeutic dose may create undesirable side effects. The combination therapy provides for a therapeutic effect with less toxicity.

The subject methods are useful for both prophylacetic and therapeutic purposes. Thus, as used herein, the term “treating” is used to refer to both prevention of disease, and treatment of a pre-existing condition. The treatment of ongoing disease, to stabilize or improve the clinical symptoms of the patient, is a particularly important benefit provided by the present invention. Such treatment is desirably performed prior to loss of function in the affected tissues; consequently, the prophylacetic therapeutic benefits provided by the invention are also important. For example, treatment of a cancer patient may be reduction of tumor size, elimination of malignant cells, prevention of metastasis, or the prevention of relapse in a patient who has been cured.

HIF-1 inhibitors. Hypoxia-inducible factor-1 (HIF1) is a transcription factor found in mammalian cells cultured under reduced oxygen tension that plays an essential role in cellular and systemic homeostatic responses to hypoxia. HIF1 is a heterodimer composed of a 120-kD HIF1-alpha subunit complexed with a 91- to 94-kD HIF1-beta subunit. Inhibitors of HIF-1 may be administered at a concentration effective in preventing the mitochondrial response to HIF-1 shown herein.

Suitable HIF-1 inhibitors include, without limitation, echinomycin (NSC-13502) (Kong et al., Cancer Research 2005 65(19): 9047-9055), which inhibits the interaction between HIF and DNA; chetomin (Kung et al., Cancer Cell 2004 6(3): 33-43), which inhibits the interaction between HIF and p300; YC-1 (3-(5′-hydroxymethyl-2′-furyl)-1-benzylindazole) (Yeo et al. Journal of the National Cancer Institute 2003 95(7): 516-525); 103D5R (Tan et al., Cancer Research 2005 65: 605-612); PX-478 (S-2-amino-3-[4V-N,N,-bis(2-chloroethyl)amino]-phenyl propionic acid N-oxide dihydrochloride) (Welsh et al., Molecular Cancer Therapeutics 2004 3: 233-244); Quinocarmycin monocitrate (KW2152) and its hydrocyanization product DX-52-1 (NSC-607097) (Rapisarda et al. Cancer Research 2002 62(15): 43164324 2002); FK228 (FR901228) (NSC 630176) (histone deacetylase inhibitor) (Mie-Lee et al., Biochemical and Biophysical Research Communications 2003 300(1): 241-246).

Such agents can be used at their known effective concentrations. Where the agent is echinomycin, the dose range is at least about 1 μg/kg, usually at least about 10 μg/kg, at least about 50 μg/kg, and not more than about 10 mg/kg, usually not more than about 1 mg/kg. Where the agent is other than echinomycin, the concentration will provide equivalent activity to such concentrations of echinomycin.

Other agents that inhibit HIF-1 function include heat shock protein-90 inhibitors, such as geldanamycin; 17-allylamino-17-demethoxygeldanamycin (17-AAG); 17-dimethylaminoethylamino-17-demethoxy-geldanamycin (17-DMAG); radicicol derivative, KF58333 (Kurebayashi et al., Cancer Research 2001 92:1342-1351).

HIF-1 inhibitors also include camptothecin analogues (topoisomerase I inhibitors) (Rapisarda et al., Cancer Research 2002 62(15): 4316-4324), e.g. Topotecan (NSC-609699), Camptothecin, 20-ester(S) (NSC-606985), and. 9-glycineamido-20(S)-camptothecin HCl (NSC-639174). Microtubule disrupting agents (Escuin et al., Cancer Research 2005 65(19): 9021-9028), such as Taxotere, 2-Methoxyestradiol, Vincristine, Discodermolide, and Epothilone B are HIF-1 inhibitors. Thioredoxin inhibitors (Welsh et al., Molecular Cancer Therapeutics 2003 2:235-243), such as PX-12 (1-methylpropyl 2-imidazolyl disulfide) and Pleurotin are HIF-1 inhibitors. Other HIF-1 inhibitors include mTOR inhibitors (Majumder et al., Nature Medicine 2004 10: 594-601), e.g. rapamycin, CCI-779, and Rad001; PI3-Kinase Inhibitors (Jiang et al., Cell Growth and Differentiation 2001 12: 363-369), e.g. wortmanin and LY294002; and polymamides targeting the hypoxia response element (Olenyuk et al, Proc Natl Acad Sci USA 2004; 101: 16768-16773).

PDK Inhibitors. The pyruvate dehydrogenase (PDH) complex is a mitochondrial multienzyme complex that catalyzes the oxidative decarboxylation of pyruvate and is one of the major enzymes responsible for the regulation of homeostasis of carbohydrate fuels in mammals. The enzymatic activity is regulated by a phosphorylation/dephosphorylation cycle. Phosphorylation of PDH by a specific pyruvate dehydrogenase kinase (PDK) results in inactivation. Inhibitors of PDK may be administered at a concentration effective in preventing the mitochondrial response to PDK shown herein.

Known inhibitors of PDK include, without limitation, pyruvate and DCA (Pratt and Roche (1979) JBC); ADP and ATP analogues (Jackson et al., Biochem, J. 1998) 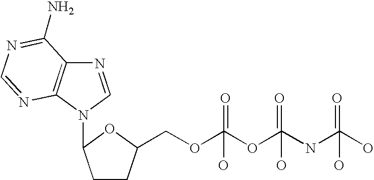
halogenated acetophenones, e.g. 2,2 dichloroacetophenone (Espinal et al., Drug Dev. Res. 1995), for example 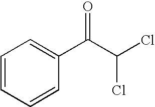
Amides of (S)-3,3,3-trifluoro-2-hydroxy-2-methylpropionic acid (Aichler et al., J. Med. Chem. 1999), for example 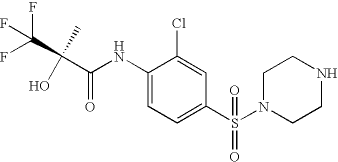
triterpene and diterpene derivatives, e.g. nortestosterone, dehydroabietyl amine (Aicher et al., Bioorg. Med. Chem. Letters, 1999); Secondary amides of (R)-3,3,3-trifluoro-2-hydroxy-2-methylpropionic acid (Aichler et al., J. Med. Chem. 2000) for example: 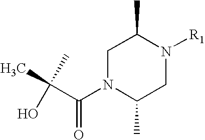
histidine modifying agents (Mooney et al., BBRC 2000), including diethyl pyrocarbonate (DEPC) and dichloro-(2,2′:6′,2″-terpyridine)-platinum(II) dehydrate (DTPD).

Such agents can be used at their known effective concentrations. Where the agent is DCA, the dose range is at least about 0.1 mg/kg, usually at least about 10 mg/kg, at least about 50 mg/kg, and not more than about 1 g/kg, usually not more than about 100 mg/kg. Where the agent is other than DCA, the concentration will provide equivalent activity to such concentrations of DCA.

Bioreductive Drugs (hypoxic cytotoxins) are cytotoxic agents that are active under hypoxic conditions, usually cytotoxic agents that are converted to an active form under hypoxic conditions. The presence of hypoxic cells in solid tumors represents an exploitable difference between normal and neoplastic tissues. One approach has been to develop bioreductive drugs, or hypoxia specific cytotoxins. These compounds exist as non-toxic prodrugs, which are only converted to their cytotoxic form under hypoxic conditions (via enzymatic bioreduction).

Classes of these drugs are known and used in the art, including quinones based on the indolequinone nucleus, e.g. Mitomycin C, EO9, Porfiromycin, etc.; nitroheterocyclic compounds, e.g. CB1954, SN23862, RSU1069 (RB6145), etc.; the benzotriazine di-N-oxide class of hypoxic cytotoxins, e.g. Tirapazamine, etc.; aliphatic N-oxides, e.g. AQ4N; and the like. See, for example, Stratford I et al., Seminars in Radiation Oncology 2003, 13(1): 42-52; and Seddon B et al., Methods in Molecular Medicine 2004, 90: 515-542, each specifically incorporated by reference.

### Disease Conditions

Cancer, as used herein, refers to hyperproliferative conditions, which for the methods of the invention are typically solid tumors. The term denotes malignant as well as non-malignant cell populations. Such disorders have an excess cell proliferation of one or more subsets of cells, which often appear to differ from the surrounding tissue both morphologically and genotypically. The excess cell proliferation can be determined by reference to the general population and/or by reference to a particular patient, e.g. at an earlier point in the patient's life. Hyperproliferative cell disorders can occur in different types of animals and in humans, and produce different physical manifestations depending upon the affected cells. The host, or patient, may be from any mammalian species, e.g. primate sp., particularly humans; rodents, including mice, rats and hamsters; rabbits; equines, bovines, canines, felines; etc. Animal models are of interest for experimental investigations, providing a model for treatment of human disease.

Tumors of interest include carcinomas, e.g. colon, prostate, breast, melanoma, ductal, endometrial, stomach, dysplastic oral mucosa, invasive oral cancer, non-small cell lung carcinoma, transitional and squamous cell urinary carcinoma, etc.; neurological malignancies, e.g. neuroblastoma, gliomas, etc.; and the like.

Some cancers of particular interest include non-small cell lung carcinoma. Non-small cell lung cancer (NSCLC) is made up of three general subtypes of lung cancer. Epidermoid carcinoma (also called squamous cell carcinoma) usually starts in one of the larger bronchial tubes and grows relatively slowly. The size of these tumors can range from very small to quite large. Adenocarcinoma starts growing near the outside surface of the lung and may vary in both size and growth rate. Some slowly growing adenocarcinomas are described as alveolar cell cancer. Large cell carcinoma starts near the surface of the lung, grows rapidly, and the growth is usually fairly large when diagnosed. Other less common forms of lung cancer are carcinoid, cylindroma, mucoepidermoid, and malignant mesothelioma.

The majority of breast cancers are adenocarcinoma subtypes. Ductal carcinoma in situ is the most common type of noninvasive breast cancer. In DCIS, the malignant cells have not metastasized through the walls of the ducts into the fatty tissue of the breast. Infiltrating (or invasive) ductal carcinoma (IDC) has metastasized through the wall of the duct and invaded the fatty tissue of the breast. Infiltrating (or invasive) lobular carcinoma (ILC) is similar to IDC, in that it has the potential metastasize elsewhere in the body. About 10% to 15% of invasive breast cancers are invasive lobular carcinomas.

Melanoma is a malignant tumor of melanocytes. Although most melanomas arise in the skin, they also may arise from mucosal surfaces or at other sites to which neural crest cells migrate. Melanoma occurs predominantly in adults, and more than half of the cases arise in apparently normal areas of the skin. Prognosis is affected by clinical and histological factors and by anatomic location of the lesion. Thickness and/or level of invasion of the melanoma, mitotic index, tumor infiltrating lymphocytes, and ulceration or bleeding at the primary site affect the prognosis. Clinical staging is based on whether the tumor has spread to regional lymph nodes or distant sites. For disease clinically confined to the primary site, the greater the thickness and depth of local invasion of the melanoma, the higher the chance of lymph node metastases and the worse the prognosis. Melanoma can spread by local extension (through lymphatics) and/or by hematogenous routes to distant sites. Any organ may be involved by metastases, but lungs and liver are common sites.

Neurologic tumors are classified according to the kind of cell from which the tumor seems to originate. Diffuse, fibrillary astrocytomas are the most common type of primary brain tumor in adults. These tumors are divided histopathologically into three grades of malignancy: World Health Organization (WHO) grade II astrocytoma, WHO grade III anaplastic astrocytoma and WHO grade IV glioblastoma multiforme (GBM). WHO grade II astocytomas are the most indolent of the diffuse astrocytoma spectrum. Astrocytomas display a remarkable tendency to infiltrate the surrounding brain, confounding therapeutic attempts at local control. These invasive abilities are often apparent in low-grade as well as high-grade tumors.

Glioblastoma multiforme is the most malignant stage of astrocytoma, with survival times of less than 2 years for most patients. Histologically, these tumors are characterized by high proliferation indices, endothelial proliferation and focal necrosis. The highly proliferative nature of these lesions likely results from multiple mitogenic effects. One of the hallmarks of GBM is endothelial proliferation. A host of angiogenic growth factors and their receptors are found in GBMs.

The compounds described herein are useful in the treatment of individuals suffering from the conditions described above, by administering an effective combined dose of a HIF-1 or PDK inhibitor in a pharmaceutical formulation, with a hypoxic cytotoxin. Diagnosis of suitable patients may utilize a variety of criteria known to those of skill in the art.

### Methods of Use

A combined therapy of inhibitor and hypoxic cytotoxin is administered to a host suffering from a hyperproliferative disorder. Administration may be topical, localized or systemic, depending on the specific disease. The compounds are administered at a combined effective dosage that over a suitable period of time substantially reduces the cellular proliferation, while minimizing any side-effects. Where the targeted cells are tumor cells, the dosage will usually kill at least about 25% of the tumor cells present, more usually at least about 50% killing, and may be about 90% or greater of the tumor cells present. It is contemplated that the composition will be obtained and used under the guidance of a physician for in vivo use. The methods may also find use in combination with non-chemotherapeutic cancer treatment, e.g. radiation, surgery, and the like, as known in the art.

To provide the synergistic effect of a combined therapy, the inhibitors can be delivered together or separately, and simultaneously or at different times within the day. In one embodiment of the invention, the inhibitor compounds are delivered prior to administration of the hypoxic cytotoxin. In one embodiment of the invention, a co-formulation is used, where the two components are combined in a single suspension. Alternatively, the two may be separately formulated.

The susceptibility of a particular tumor cell to killing with the combined therapy may be determined by in vitro testing, as detailed in the experimental section. Typically a culture of the tumor cell is combined with a combination of inhibitor and a hypoxic cytotoxin at varying concentrations for a period of time sufficient to allow the active agents to induce cell killing. For in vitro testing, cultured cells from a biopsy sample of the tumor may be used. The viable cells left after treatment are then counted.

The dose will vary depending on the specific hypoxic cytotoxin utilized, type of cells targeted by the treatment, patient status, etc., at a dose sufficient to substantially ablate the targeted cell population, while maintaining patient viability.

The inhibitors can be incorporated into a variety of formulations for therapeutic administration. Part of the total dose may be administered by different routes. Such administration may use any route that results in systemic absorption, by any one of several known routes, including but not limited to inhalation, i.e. pulmonary aerosol administration; intranasal; sublingually; orally; and by injection, e.g. subcutaneously, intramuscularly, etc.

For injectables, the agents are used in formulations containing cyclodextrin, cremophor, DMSO, ethanol, propylene glycol, solutol, Tween, triglyceride and/or PEG. For oral preparations, the agents are used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and in some embodiments, with diluents, buffering agents, moistening agents, preservatives and flavoring agents.

Formulations are typically provided in a unit dosage form, where the term “unit dosage form,” refers to physically discrete units suitable as unitary dosages for human subjects, each unit containing a predetermined quantity of inhibitor calculated in an amount sufficient to produce the desired effect in association with a pharmaceutically acceptable diluent, carrier or vehicle. The specifications for the unit dosage forms of the present invention depend on the particular complex employed and the effect to be achieved, and the pharmacodynamics associated with each complex in the host.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Depending on the patient and condition being treated and on the administration route, the inhibitor is administered in dosages of 0.1 mg to 2000 mg/kg body weight per day, e.g. about 100, 500, 1000, 10,000 mg/day for an average person. Durations of the regimen may be from: 1×, 2× 3× daily; and in a combination regimen may be from about 1, about 7, about 14, etc. days prior to administration of second agent. Dosages are appropriately adjusted for pediatric formulation. Those of skill will readily appreciate that dose levels can vary as a function of the specific inhibitor, the diet of the patient and the gluten content of the diet, the severity of the symptoms, and the susceptibility of the subject to side effects. Some of the inhibitors of the invention are more potent than others. Preferred dosages for a given inhibitor are readily determinable by those of skill in the art by a variety of means. A preferred means is to measure the physiological potency of a given compound.

Various methods for administration are employed in the practice of the invention. The dosage of the therapeutic formulation can vary widely, depending upon the nature of the disease, the frequency of administration, the manner of administration, the clearance of the agent from the patient, and the like. The initial dose can be larger, followed by smaller maintenance doses. The dose can be administered as infrequently as weekly or biweekly, or more often fractionated into smaller doses and administered daily, with meals, semi-weekly, and the like, to maintain an effective dosage level.

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature), but some experimental errors and deviations may be present. Unless indicated otherwise, parts are parts by weight, molecular weight is weight average molecular weight, temperature is in degrees Centigrade, and pressure is at or near atmospheric.

General methods in molecular and cellular biochemistry can be found in such standard textbooks as Molecular Cloning: A Laboratory Manual, 3rd Ed. (Sambrook et al., Harbor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998). Reagents, cloning vectors, and kits for genetic manipulation referred to in this disclosure are available from commercial vendors such as BioRad, Stratagene, Invitrogen, Sigma-Aldrich, and ClonTech.

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

### EXAMPLES

### Example 1

Materials and Methods

Cell lines and cell culture. Primary human fibroblasts have been described previously (Denko et al., 2003; Kim et al., 1997) and RKO human colon carcinoma cells were obtained from the American Type Culture Collection (ATCC, Manassas, Va.). RCC4, RCC4/VHL, and RCC4/Y98H human renal cell carcinoma cell lines have also been described previously (Chan et al., 2005). Wildtype and HIF-1α knockout mouse embryo fibroblasts (MEFs) were a gift from Dr. R Johnson (University of California, San Diego). All cells were grown in vitro as monolayers in Dulbeco's Modified Eagle's Media (DMEM) supplemented with 10% fetal bovine serum (FBS). For treatment with moderate hypoxia, cell culture dishes were placed into an Invivo2 humidified hypoxia workstation (Ruskinn Technologies, Bridgend, UK) at the indicated oxygen concentrations. Severe hypoxia was generated in an anaerobic workstation with a palladium catalyst (Sheldon Co., Cornelius, Oreg.). Tirapazamine toxicity was measured in cells plated overnight at the indicated density in glass dishes. The next day, fresh media was added containing the indicated concentration of TPZ, and the cells placed in the indicated oxygen environment. 24 hours later, the cells were trypsinized, counted, and plated for colony formation in normoxia.

Plasmids and siRNAs. Full length human PDK1 cDNA was obtained from the mammalian gene collection (MGC), through the ATCC, and was cloned into pEF2aIRESpuro creating pPDK1IRESpuro. pDsRed2-mito was constructed by cloning a 28 amino acid mitochondrial targeting sequence from human cytochrome c oxidase subunit VIII into the pDsRed2-N1 vector (Clontech, Mountain View, Calif.). For knockdown experiments, the target sequences used were: HIF-1α, (SEQ ID NO:1) UGAGGAAGUACCAUUAUAU and PDK1, (SEQ ID NO:2) CGACACMUGAUGUCAUUCCCACAA. For construction of stable knockdown cell lines, the sequences listed above were cloned into pSUPER using synthetic 64mer oligonucleotides (Brummelkamp et al., 2002). RKO/shPDK1 and RKO/shHIF-1α stable knockdown cells were created by co-transfecting RKO cells with pTKhygro, and either empty pSuper, pSuper-shPDK1 or pSuper-shHIF-1α (1:20 ratio) using Lipofectamine (Invitrogen, Carlsbad, Calif.), followed by selection in 500 ug/ml hygromycin. The RKOPDK1IRESpuro stable overexpressing cell lines were established by transfecting RKO cells with pPDK1IRESpuro using Lipofectamine followed by culture in 2 μg/ml puromycin.

Microarray Analysis. Total RNA was extracted using TriZol reagent (Invitrogen). Preparation of cDNA and cRNA was conducted following instructions in the Affymetrix GeneChip Expression Analysis Manual (Affymetrix, Santa Clara, Calif.). cRNA was hybridized to an oligo-based array, washed and scanned according to standard Affymetrix protocols. Data from the scanning of the Affymetrix GeneChips was gathered using the Affymetrix Microarray Suite v4.0 and exported to Microsoft Excel.

Oxygen consumption measurements. Cells were trypsinized and suspended at 3×10⁶ to 6×10⁶ cells per ml in normoxic DMEM+10% FBS. Oxygen consumption was measured in a 0.5 ml volume using an Oxytherm electrode unit (Hansatech, Norfolk, UK). This system employs a Clark type oxygen electrode to monitor the dissolved oxygen concentration in a sealed measurement chamber over time. The data are exported to a computerized chart recorder (Oxygraph 1.01, Hansatech, Norfolk, UK), which is used to calculate the rate of oxygen consumption. A small stir bar maintains the cells in suspension for the duration of the measurements, and a peltier heating block maintains the temperature at 37° C. Since the electrode consumes oxygen during measurement, the rate of oxygen drop in 0.5 ml of DMEM media without cells was established and subtracted from the total oxygen consumption rates for the cell suspensions.

Western Blotting. Briefly, treated cells were harvested directly in RIPA buffer containing protease inhibitors, protein concentrations were quantitated, 25-50 μg were electrophoresed on a reducing Tris-Tricine gel and electroblotted to PVDF membrane. Antibodies used were murine α-HIF-1 Transduction Labs (1:1000), rabbit α-PDK1 Stressgen (1:2000), murine α-alpha tubulin Research Diagnostics (1:2000) goat α-HSP60 Santa Cruz (1:2000), rabbit α-Bnip3 and a Bnip3L were described previously (1:500) (Papandreou et al., 2005a). Primary anitibodies were detected with species-specific secondary antibodies labeled with Alkaline Phosphatase (Vector labs 1:3000) and visualized with ECF (Amersham) on a Storm 860 phophoimager (Molecular Devices).

Mitochondrial membrane potential staining and flow cytometry. Rhodamine 123 (R123) (Molecular Probes, Eugene, Oreg.) uptake was used to measure mitochondrial membrane potential. Cells were trypsinized, counted, and suspended at 5×10⁵ cells per ml in DMEM+10% FBS with 10 μg/ml R123, at 37° C. for 10 min. The cells were pelleted by centrifugation, resuspended in cold DMEM+10% FBS. Fluorescence was measured in a FACSCalibur flow cytometer (Becton Dickinson, San Jose, Calif.). Forward scatter and side scatter values were used to gate on whole cells, and R123 fluorescence was measured for 10000 cells per sample using the FL2 channel. After instrument setup, all gain and amplifier settings were held constant for the duration of the experiment. Since the R123 fluorescence values for all samples displayed a log normal distribution, the geometric mean was used as a quantitative measure of the cell population's membrane potential.

Immunocytochemistry and fluorescence microscopy. Cells were plated on glass multiwell chamber slides (Nalge Nunc, Naperville, Ill.) in DMEM+10% FBS. For examination of mitochondrial DsRed2, cells were transfected with pDsRed2-mito using Lipofectamine. After 48 hours, the cells were fixed in 4% paraformaldehyde, and mounted in Vectashield (Vector Laboratories, Burlingame, Calif.). For cytochrome c immunocytochemistry, chamber slides were fixed in 4% paraformaldehyde, blocked in PBS Tween milk (0.2% Tween 20, 5% non-fat dry milk, in PBS) overnight at 4° C. Cytochrome c was visualized using a mouse monoclonal primary antibody 1:250 (BD Pharmingen, San Diego, Calif.) and an anti-mouse Alexa 488 secondary antibody 1:500 (Molecular Probes). Cells were visualized on a Nikon Eclipse E800 microscope, with a Spot RT Slider CCD digital camera using standard FITC and Texas Red filter blocks (Diagnostic Instruments, Sterling Heights, Mich.).

Pimonidazole Staining. Hypoxyprobe 1 (pimonidazole hydrochloride) was purchased from Chemicon (Temecula, Calif.). Cells were treated at 60 mg/L in the culture medium during the final 4 hours of the hypoxic exposure, harvested, fixed in 4% paraformaldehyde, blocked in PBS containing 5% non-fat dry milk and 0.1% Triton and 4% FBS (PBS-T-milk-FBS). After 2 hours blocking, cells were treated with FITC-labeled anti-pimonidozole mAb at 1:25 dilution for 2 hours in PBS-T-milk-FBS. The cells were then washed in PBS-T-milk-FBS and relative FITC was measured on channel 1 in a Beckton-Dickenson FacScanner.

Data Analysis. Oxygen consumption experiments were repeated three times in duplicate, survival was measured three times in triplicate, reporter assays were repeated two times in quadruplicate. Error bars represent the standard error of the mean.

Results

Identification of HIF-dependent mitochondrial proteins through genomic and bioinformatics approaches. In order to elucidate the role of HIF-1α in regulating metabolism, we undertook a genomic search for genes that were regulated by HIF-1 in tumor cells exposed to hypoxia in vitro. We used genetically matched human RCC4 cells that had lost VHL during tumorigenesis and displayed constitutive HIF-1 activity, and a cell line engineered to re-express VHL to establish hypoxia-dependent HIF activation. These cells were treated with 24 hours of stringent hypoxia (<0.01% oxygen), and microarray analysis performed. Using a strict 2.5 fold elevation as our cutoff, we identified 173 genes that were regulated by hypoxia and/or VHL status (Table 1). We used the pattern of expression in these experiments to identify putative HIF-regulated genes; ones that were constitutively elevated in the parent RCC4s independent of hypoxia, downregulated in the RCC4VHL cells under normoxia and elevated in response to hypoxia. Of the 173 hypoxia and VHL regulated genes, 74 fit the putative HIF-1 target pattern. The open reading frames of these genes were run through a pair of bioinformatics engines in order to predict subcellular localization, and 10 proteins scored as mitochondrial on at least one engine. The genes, fold induction, and mitochondrial scores are listed in table 1.

Table 1 Identification of putative HIF-1α regulated mitochondrial proteins through microarray bioinformatics and data mining approaches. Ind 1 and 2 represent the fold induction for the indicated parameter based on the two microarray experiments, Loc1 is the predicted subcellular location based on TargetP V1.0 and Loc2 is the predicted subcellular localization based on ProtComp V6.0.

Genes over-expressed in normoxia in RCC4/VHL cells as compared to RCC4 cells, but not hypoxia responsive (36)

| | Identifier | fold I | fold II |
|---|---|---|---|
| Matrix metalloproteinase 7 (MMP7) | AI675414 | 274.0 | 20.3 |
| DKFZp434L142 Hypothetical protein | AL834177 | 132.0 | 3.4 |
| ESTs | Hs.145527 | 83.0 | 4.4 |
| Homo sapiens mRNA; cDNA DKFZp313L231 | AL832465 | 82.0 | 253.0 |
| Serum deprivation response (SDPR) | BC016475 | 55.0 | 5.9 |
| ESTs | Hs.23762 | 39.0 | 2.8 |
| Matrilin 2 (MATN2) | U69263 | 37.0 | 6.0 |
| Neuroligin 1 (NLGN1) | AB028993 | 28.0 | 100.0 |
| ESTs | Hs.104972 | 22.0 | 4.3 |
| Homo sapiens cDNA: FLJ22806 fis, clone KAIA2845 | AK026459 | 19.0 | 2.8 |
| ESTs | Hs.76704 | 17.2 | 7.3 |
| Bromodomain and PHD finger containing, 3 (BRPF3) | AB033112 | 15.0 | 107.0 |
| Annexin A3 (ANXA3) | M20560 | 11.0 | 118.0 |
| Cyclin D2 (CCND2) | D13639 | 8.1 | 15.1 |
| Caspase 4 (CASP4) | U28976 | 8.0 | 3.9 |
| ESTs | Hs.187447 | 7.5 | 3.6 |
| Homo sapiens cDNA FLJ34764 fis, clone NT2NE2002311 | AK092083 | 7.1 | 3.2 |
| I factor (IF) | J02770 | 7.1 | 12.7 |
| E-cadherin | 6.6 | 4.2 | |
| Klotho (KL) | AB005142 | 5.7 | 5.8 |
| Unigene cluster containing H factor (complement)-like 1/2/3 | Hs.194272 | 5.7 | 5.8 |
| Fibronectin 1 (FN1) | X02761 | 5.6 | 3.6 |
| Secreted frizzled-related protein 4 (SFRP4) | AF026692 | 4.8 | 4.4 |
| ESTs | Hs.48713 | 4.7 | 10.2 |
| Cingulin (CGN) | AF263462 | 4.7 | 3.2 |
| C1S Complement component 1, C2 (CFTR/MRP) | 4.1 | 10.4 | |
| Fibronectin, Alt. Splice 1 | 4.1 | 3.0 | |
| ESTs | Hs.108977 | 3.9 | 2.6 |
| Homo sapiens cDNA clone EUROIMAGE 1913076 | AL359062 | 3.9 | 2.7 |
| Transmembrane 4 superfamily member 4 (TM4SF4) | U31449 | 3.8 | 4.1 |
| hypothetical protein FLJ32122 | AK056684 | 3.2 | 2.9 |
| Unigene cluster containing Albumin (ALB) + (C3) | Hs.58512 | 3.2 | 8.5 |
| Thiamine pyrophosphokinase (TPK1) | AF297710 | 3.0 | 2.7 |
| Cardiac ankyrin repeat protein (CARP) | X83703 | 2.9 | 2.5 |
| Homo sapiens mRNA; cDNA DKFZp564O0862 | AL080095 | 2.9 | 7.1 |
| DKFZP586A0522 protein | AK023693 | 5.5 | 6.0 |

Genes over-expressed in normoxia in RCC4 cells but not induced under hypoxia in RCC4/VHL cells (49)

| | | vhl−/vhl+ | |
|---|---|---|---|
| Identifier | vhl−/vhl+ fold I | fold II | |
| PDZ domain containing 3 (PDZK3) | AF338650 | 114.0 | 369.0 |
| Homo sapiens cDNA: FLJ21962 fis, clone | AK025615 | 58.0 | 355.0 |
| HEP05564 | | | |
| HIF-prolyl-hydroxylase 3 (PDH3) | AK025273 | 48.0 | 242.0 |
| KIAA1750 protein | AB051537 | 41.0 | 199.0 |
| Homo sapiens cDNA clone IMAGE: 7958 5′, mRNA | 36.0 | 7.0 | |
| sequence | | | |
| Homo sapiens cDNA FLJ40582 fis, clone | BC045584 | 29.0 | 130.0 |
| THYMU2007886 | | | |
| CASP8 and FADD-like apoptosis regulator | Y14039 | 26.0 | 5.3 |
| (CASPER) | | | |
| C14orf75 chromosome 14 open reading frame 75 | Hs.21454 | 17.0 | 19.3 |
| Friend of EBNA2 (FOE) | AF479418 | 15.0 | 4.6 |
| KIAA1909 protein | AB067496 | 12.0 | 117.0 |
| SLBP Stem-loop (histone) binding protein | U75679 | 11.0 | 3.3 |
| Unigene cluster | Hs.11356 | 10.0 | 112.0 |
| Homo sapiens, clone IMAGE: 5240524, mRNA | BC038798 | 9.0 | 138.0 |
| Enhancer of filamentation 1 (HEF1) | L43821 | 8.7 | 113.0 |
| Homo sapiens mRNA; cDNA DKFZp586F2224 | AL110157 | 8.0 | 21.7 |
| Hypothetical protein FLJ20220 | AK000227 | 7.0 | 4.9 |
| Carbonic anhydrase IX | X66839 | 7.0 | 5.5 |
| NADH: ubiquinone oxidoreductase MLRQ subunit | BC011910 | 6.4 | 3.2 |
| homolog | | | |
| clone MGC: 39900 IMAGE: 5247537, mRNA, | BC028039 | 6.3 | 2.8 |
| complete cds | | | |
| FLJ25477 Hypothetical protein | AK098343 | 5.5 | 2.5 |
| MOT8 Hypothetical protein | AF175409 | 5.0 | 35.3 |
| ESTs | Hs.9403 | 5.0 | 3.2 |
| glutathione peroxidase 6 | AK027683 | 5.0 | 146.0 |
| Glucocorticoid receptor DNA binding factor 1 | AB051509 | 4.75 | 3.6 |
| (GRLF1) | | | |
| Slit (Drosophila) homolog 3 (SLIT3) | AB017169 | 4.2 | 6.1 |
| phosphatidylinositol 4-kinase type-II beta (PI4K2B) | AY065990 | 4.1 | 3.6 |
| Cyclin D1 (CCND1) | X59798 | 3.7 | 3.7 |
| KIAA0843 protein | AB020650 | 3.7 | 3.2 |
| Decidual protein induced by progesterone (DEPP) | AB022718 | 3.7 | 6.2 |
| Fatty acid binding protein 6 (FABP6) | X90908 | 3.6 | 4.4 |
| WAS protein family, member 3 (WASF3) | AB026543 | 3.2 | 2.6 |
| DnaJ (Hsp40) homolog, subfamily C, member 9 | AF327347 | 3.0 | 2.7 |
| Neural cell expressed, developmentally down- | D42055 | 3.0 | 2.6 |
| regulated (NEDD4) | | | |
| hypothetical protein LOC144100 | BC033239 | 3.0 | 3.4 |
| Metallo phosphoesterase (MPPE1) | AF363484 | 3.0 | 4.7 |
| Prostaglandin-endoperoxide synthase 2 (PTGS2) | U04636 | 2.9 | 3.4 |
| Phosphorylase kinase, alpha 2 (PHKA2) | X80497 | 2.9 | 2.7 |
| Homo sapiens, clone IMAGE: 4564684, mRNA, | BC014203 | 2.9 | 3.3 |
| partial cds | | | |
| Kinase suppressor of ras (KSR) | U43586 | 2.8 | 5.1 |
| Cytochrome b-561 (CYB561) | BC002976 | 2.8 | 4.2 |
| UDP glycosyltransferase 8 (UGT8) | U62899 | 2.8 | 2.9 |
| Neuron navigator 1 (NAV1) | AY043013 | 2.7 | 3.2 |
| KIAA0595 protein | AF325193 | 2.7 | 6.3 |
| Putative small membrane protein NID67 | AF313413 | 2.6 | 3.3 |
| Potassium channel calcium activated, member 1 | U11058 | 2.6 | 2.7 |
| (KCNMA1) | | | |
| gap junction protein, beta 2, 26 kD (connexin 26) | 2.6 | 3.3 | |
| Paraneoplastic antigen MA2 (PNMA2) | AB020690 | 2.5 | 4.6 |
| Hypothetical protein FLJ20335 | AF399753 | 2.5 | 2.9 |
| hypothetical protein LOC255326 | BC041413 | 2.5 | 4.8 |

Genes overexpressed in normoxia in RCC4 cells and induced by hypoxia in RCC4/VHL cells

| Identifier | fold I | fold II | |
|---|---|---|---|
| KIAA1376 protein | BC015928 | 118.0 | 56.0 |
| CA12 Carbonic anhydrase XII | AF037335 | 96.0 | 80.0 |
| GBE1 glycogen branching enzyme | L07956 | 96.0 | 4.5 |
| MXI1 MAX-interacting protein 1 | L07648 | 66.0 | 135.0 |
| PYGL Phosphorylase, glycogen | Y15233 | 60.0 | 3.6 |
| Cyclin G2 | U47414 | 56.0 | 9.2 |
| HSU79274 Protein predicted by clone 23733 | U79274 | 50.0 | 65.0 |
| VEGF | AF022375 | 49.0 | 41.0 |
| LOC51141 Insulin induced protein 2 | AF125392 | 43.3 | 103.9 |
| Semaphorin 4B | AB051532 | 43.0 | 31.0 |
| EST | Hs.8705 | 37.0 | 11.0 |
| CKLiK CamKI-like protein kinase | AF286366 | 25.0 | 96.0 |
| LOX Lysyl Oxidase | AF039291 | 24.0 | 5.1 |
| Homo sapiens mRNA; cDNA DKFZp686M2414 | AL832164.1 | 23.0 | 32.0 |
| Homo sapiens cDNA FLJ11157 fis, clone | AK002019.1 | 20.0 | 2.9 |
| PLACE1006961 | | | |
| RIS1 Ras-induced senescence 1 | AF438313 | 19.8 | 2.9 |
| DEC2 | AB044088 | 18.0 | 29.0 |
| KMO Kynurenine 3-monooxygenase | AF056032 | 18.0 | 6.0 |
| TSPAN-2 Tetraspan 2 | AK022144 | 16.2 | 2.7 |
| LOC51754 NAG-5 protein | AF188239 | 16.0 | 5.1 |
| EST | BC013423 | 15.0 | 4.3 |
| Epican, Alt. Splice 1 | 14.0 | 56.0 | |
| SLC9A6 sodium/hydrogen exchanger, isoform 6 | AF030409 | 13.0 | 19.0 |
| EST | Hs.55272 | 13.0 | 19.0 |
| PER2 period homolog 2 | AB002345 | 11.7 | 48.5 |
| Immunoglobulin Recombination Signal Sequence | 11.0 | 4.7 | |
| BP | | | |
| ALS2CR9 amyotrophic lateral sclerosis 2, | AB053311 | 11.0 | 3.0 |
| candidate 9 | | | |
| Hypothetical protein LOC202451 | AK056626 | 10.5 | 5.8 |
| GPT2 Glutamic pyruvate transaminase (alanine | AY029173 | 10.0 | 10.0 |
| aminotransferase) 2 | | | |
| ADM Adrenomedullin | D14874 | 9.7 | 6.6 |
| BNIP3 BCL2/adenovirus E1B 19 kD-interacting | AF002697 | 9.1 | 4.3 |
| protein 3 (NIP3) | | | |
| Homo sapiens cDNA FLJ36681 fis, clone | AK094000 | 9.0 | 45.0 |
| UTERU2006547 | | | |
| KLF7 Kruppel-like factor 7 | AB015132 | 9.0 | 31.0 |
| KIAA0779 protein | AB018322 | 9.0 | 4.0 |
| Homo sapiens cDNA FLJ36544 fis, clone | AK093863 | 9.0 | 3.3 |
| TRACH2006378 | | | |
| CGI-116 CGI-116 protein | AF151874 | 8.0 | 39.0 |
| SSBP2 Single-stranded DNA-binding protein 2 | AL080076 | 7.5 | 4.5 |
| Integrin, beta 7 | S80335 | 7.3 | 4.0 |
| PPP1R3C Protein phosphatase 1, regulatory | BC012625 | 7.3 | 6.9 |
| (inhibitor) subunit 3C | | | |
| ADARB1 adenosine deaminase, RNA-specific, B1 | U76420-U76422 | 7.0 | 6.0 |
| neuritin 1 | AF136631 | 6.8 | 5.2 |
| transforming growth factor; alpha | X70340 | 6.7 | 2.7 |
| DKFZp761O0113 hypothetical protein | AL161975 | 6.5 | 4.5 |
| FLJ11200 Hypothetical protein | AK002062 | 5.2 | 4.1 |
| Homo sapiens cDNA: FLJ22448 fis, clone | Hs.11530 | 5.0 | 58.0 |
| HRC09541 | | | |
| PMAIP1 Phorbol-12-myristate-13-acetate-induced | D90070 | 5.0 | 8.1 |
| prot 1 | | | |
| KIAA1376 protein | AB037797 | 4.6 | 3.7 |
| NDRG1 N-myc downstream regulated gene 1 | X92845 | 4.6 | 2.9 |
| ESTs | Hs.186733 | 4.5 | 9.0 |
| Enolase 2 | X51956 | 4.4 | 4.6 |
| C20orf97 chromosome 20 open reading frame 97 | AK026945 | 4.3 | 3.5 |
| KIAA0870 protein | AB020677.2 | 4.2 | 34.0 |
| Ribonuclease, RNase A family/EST | BC015520 | 4.2 | 3.9 |
| P4HA2 proline 4-hydroxylase alpha polypeptide II | U90441 | 4.1 | 3.2 |
| FLJ10997 Hypothetical protein | AK001859 | 4.1 | 3.4 |
| NFIL3 Nuclear factor, interleukin 3 regulated | S79880 | 4.0 | 3.3 |
| Rag D protein | AF272036 | 4.0 | 3.3 |
| IPT TRNA isopentenyltransferase 1 | AK000068 | 3.8 | 2.8 |
| AP1G1 Adaptor-related protein complex 1, gamma | Y12226 | 3.5 | 3.2 |
| 1 subunit | | | |
| FLJ11210 Hypothetical protein | AK002072 | 3.1 | 2.8 |
| ESTs | Hs.25661 | 3.1 | 3.3 |
| PDK1 Pyruvate dehydrogenase kinase, isoenzyme 1 | L42450 | 3.1 | 5.9 |
| Homo sapiens, clone IMAGE: 4830497, mRNA | BC039121 | 3.0 | 86.0 |
| Hypothetical protein DKFZp761K1423 | AL353936 | 3.0 | 38.0 |
| Unigene cluster | Hs.179788 | 3.0 | 12.0 |
| FLJ36666 hypothetical protein FLJ36666 | AK093985 | 2.9 | 9.0 |
| RLF Rearranged L-myc fusion sequence | U22377 | 2.8 | 50.0 |
| CEBPG CCAAT/enhancer binding protein (C/EBP), | U20240 | 2.8 | 2.8 |
| gamma | | | |
| Unigene cluster | Hs.29977 | 2.7 | 2.8 |
| Homo sapiens cDNA FLJ34899 fis | AK092218 | 2.7 | 5.1 |
| LOC201164 similar to CG12314 gene product | AK090899 | 2.7 | 5.0 |
| PRSS16 Protease, serine, 16 | AA580758 | 2.7 | 2.5 |
| Homo sapiens, clone IMAGE: 4798730, mRNA | BC045797 | 2.6 | 3.5 |
| PFKFB4 phosphofructo-2-kinase/fructose-2,6- | BC010269 | 2.6 | 2.6 |
| biphosphatase 4 | | | |
| SH3GL3 SH3-domain GRB2-like 3 | AF036271 | 2.6 | 14.2 |
| PTPN14 Protein tyrosine phosphatase, non- | BC017300/X82676 | 2.5 | 3.0 |
| receptor type 14 | | | |
| Hypothetical protein FLJ21939 | AK025592 | 2.5 | 7.6 |

Genes induced by hypoxia in RCC4/VHL cells, but not over-expressed in normoxia in RCC4 cells (11)

| Accession/Unigene | fold I | fold II | vhl−/vhl+ 1 | vhl−/vhl+ 2 | |
|---|---|---|---|---|---|
| LOC115330 | BC014241 | 52.0 | 43.0 | 1 | 1 |
| hypothetical protein | | | | | |
| BC014241 | | | | | |
| hypothetical protein | AK023370 | 9.5 | 3.6 | 2.23 | 2.12 |
| FLJ10201 | | | | | |
| hypothetical protein | AF193051 | 4.5 | 2.7 | 1.58 | 1.64 |
| CLONE24945 | | | | | |
| Oncogene Tls/Chop, | 3.8 | 4.5 | 1.26 | 1.07 | |
| Fusion Activated | | | | | |
| EDN1 Endothelin 1 | BC009720 | 3.6 | 3.5 | 1.98 | 2.2 |
| E2IG5 Hypothetical protein, | AF250321 | 3.4 | 2.8 | 2.4 | 2.28 |
| estradiol-induced | | | | | |
| EFNA3 Ephrin-A3 | BC017722 | 3.3 | 3.3 | 2.02 | 1.67 |
| HIG2 Hypoxia-inducible Gene 2 | AF144755 | 2.9 | 3.7 | 1.91 | 1.62 |
| TNFAIP3 Tumor | M59456 | 2.7 | 3.9 | 1.05 | 1.19 |
| necrosis factor, alpha-induced | | | | | |
| protein 3 | | | | | |
| Homo sapiens, clone | BC033829 | 2.6 | 2.8 | 1.31 | 2.01 |
| IMAGE: 3856003, mRNA | | | | | |
| MAFF V-maf | AJ010857 | 2.6 | 2.7 | 1.8 | 2.32 |
| fibrosarcoma oncogene family, | | | | | |
| protein F | | | | | |

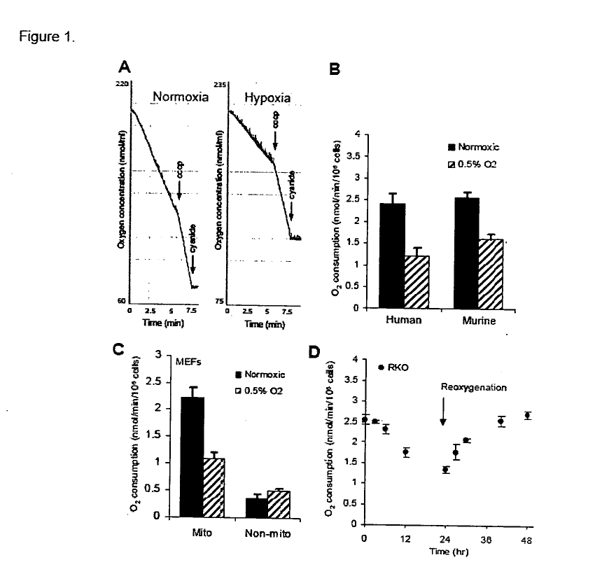

HIF-1 downregulates mitochondrial oxygen consumption. Having identified several putative HIF-1 responsive gene products that had the potential to regulate mitochondrial function, we then directly measured mitochondrial oxygen consumption in cells exposed to long term hypoxia. This is one of the first descriptions of mitochondrial function after long term hypoxia where there have been extensive hypoxia-induced gene expression changes. FIG. 1a is an example of the primary oxygen trace from a Clark electrode showing a drop in oxygen concentration in cell suspensions of primary fibroblasts taken from normoxic and hypoxic cultures. The slope of the curve is a direct measure of the total cellular oxygen consumption rate. Exposure of either primary human or immortalized mouse fibroblasts to 24 hours of hypoxia resulted in a reduction of this rate by approximately 50% (FIGS. 1a and 1b). In these experiments, the oxygen consumption can be stimulated with the mitochondrial uncoupling agent CCCP (carbonyl cyanide 3-chloro phenylhydrazone) and was completely inhibited by 2 mM potassium cyanide. We determined that the change in total cellular oxygen consumption was due to changes in mitochondrial activity by the use of the cell-permeable poison of mitochondrial complex 3, Antimycin A. FIG. 1c shows that the difference in the normoxic and hypoxic oxygen consumption in murine fibroblasts is entirely due to the Antimycin-sensitive mitochondrial consumption. The kinetics with which mitochondrial function slows in hypoxic tumor cells also suggests that it is due to gene expression changes because it takes over 6 hours to achieve maximal reduction, and the reversal of this repression requires at least another 6 hours of reoxygenation (FIG. 1d). These effects are not likely due to proliferation or toxicity of the treatments as these conditions are not growth inhibitory or toxic to the cells.

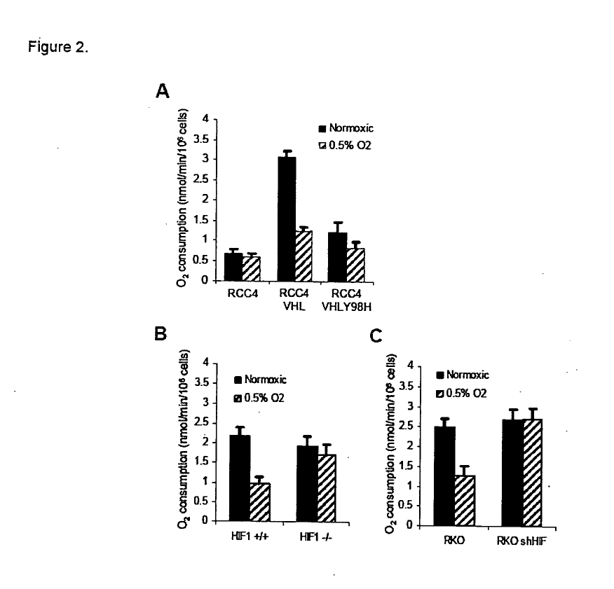

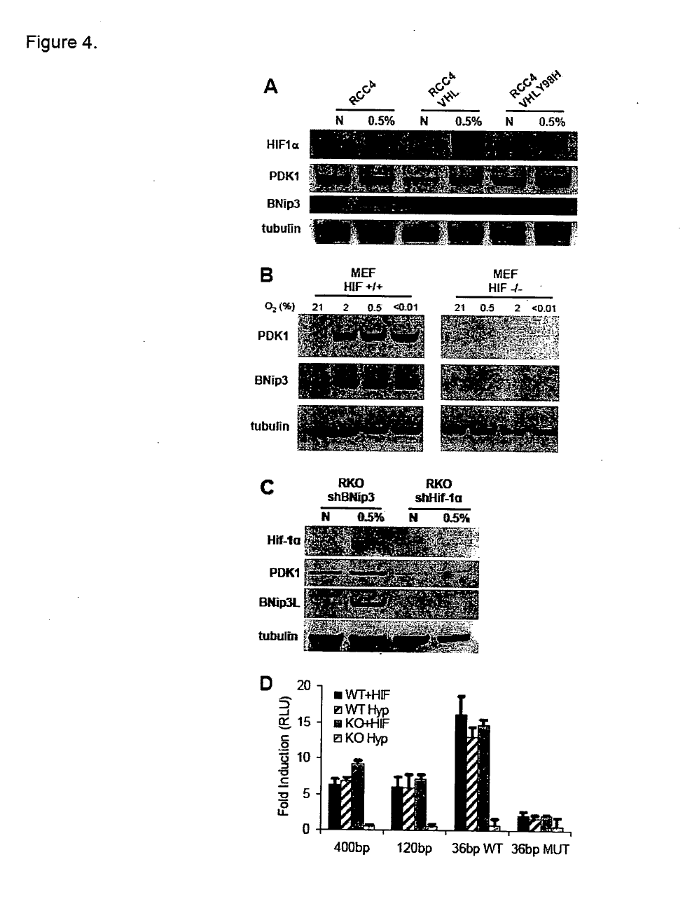

Since we had predicted from the gene expression data that the mitochondrial oxygen consumption changes were due to HIF-1 mediated expression changes, we tested several genetically matched systems to determine what role HIF-1 played in the process (FIG. 2). We first tested the cell lines that had been used for microarray analysis and found that the parental RCC4 cells had reduced mitochondrial oxygen consumption when compared to the VHL-reintroduced cells. Oxygen consumption in the parental cells was insensitive to hypoxia, while it was reduced by hypoxia in the wild-type VHL transfected cell lines. Interestingly, stable introduction of a tumor-derived mutant VHL (Y98H) that cannot degrade HIF was also unable to restore oxygen consumption. These results indicate that increased expression of HIF-1 is sufficient to reduce oxygen consumption (FIG. 2a). We also investigated whether HIF-1 induction was required for the observed reduction in oxygen consumption in hypoxia using two genetically matched systems. We measured normoxic and hypoxic oxygen consumption in murine fibroblasts derived from wild-type or HIF-1α null embryos (FIG. 2b), and from human RKO tumor cells and RKO cells constitutively expressing ShRNAs directed against the HIF-1α gene (FIGS. 2c and 4c). Neither of the HIF-deficient cell systems was able to reduce oxygen consumption in response to hypoxia. These data from the HIF overexpressing RCC cells and the HIF-deficient cells indicate that HIF-1 is both necessary and sufficient for reducing mitochondrial oxygen consumption in hypoxia.

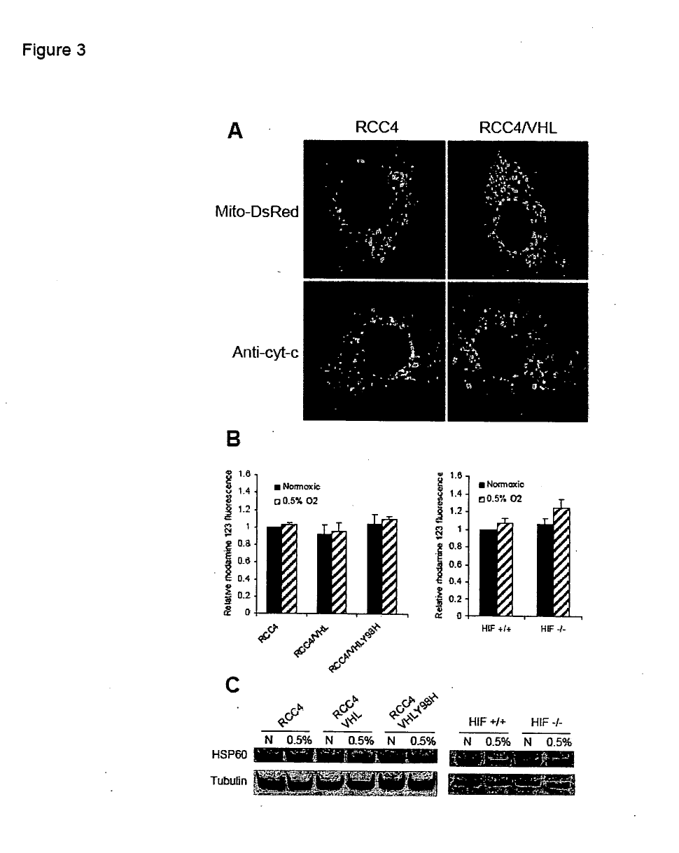

HIF-dependent mitochondrial changes are functional, not structural. Because addition of CCCP could increase oxygen consumption even in the hypoxia treated cells, we hypothesized that the hypoxic inhibition was a regulated activity, not a structural change in the mitochondria in response to hypoxic stress. We confirmed this interpretation by examining several additional mitochondrial characteristics in hypoxic cells such as mitochondrial morphology, quantity, and membrane potential. We examined morphology by visual inspection of both the transiently transfected mitochondrially-localized DsRed protein, and the endogenous mitochondrial protein cytochrome C. Both markers were indistinguishable in the parental RCC4 and the RCC4VHL cells (FIG. 3a). Likewise, we measured the mitochondrial membrane potential with the functional dye rhodamine 123, and found that it was identical in the matched RCC4 cells and the matched HIF wt and knockout cells when cultured in normoxia or hypoxia (FIG. 3b). Finally, we determined that the quantity of mitochondria per cell was not altered in response to HIF or hypoxia by showing that the amount of the mitochondrial marker protein HSP60 was identical in the RCC4 and HIF cell lines (FIG. 3c).

PDK1 is a HIF-1 inducible target protein. After examination of the list of putative HIF-regulated mitochondrial target genes, we hypothesized that PDK1 could mediate the functional changes that we observed in hypoxia. We therefore investigated PDK1 protein expression in response to HIF and hypoxia in the genetically matched cell systems. FIG. 4a shows that in the RCC4 cells PDK1 and the HIF-target gene BNip3 were both induced by hypoxia in a VHL-dependent manner, with the expression of PDK1 inversely matching the oxygen consumption measured in FIG. 1 above. Likewise the HIFwt MEFs show oxygen dependent induction of PDK1 and BNip3, while the HIFko MEFs did not show any expression of either of these proteins under any oxygen conditions (FIG. 4b). Finally, the parental RKO cells were able to induce PDK1 and the HIF target gene BNip3L in response to hypoxia, while the HIF-depleted ShRNA RKO cells could not induce either protein (FIG. 4c). Therefore, in all three cell types the HIF-1 dependent regulation of oxygen consumption seen in FIG. 2, corresponds to the HIF-1 dependent induction of PDK1 seen in FIG. 4.

In order to determine if PDK1 was a direct HIF-1 target gene, we analyzed the genomic sequence flanking the 5′ end of the gene for possible HIF-1 binding sites based on the consensus core HRE element (A/G)CGTG. Several such sites exist within the first 400 bases upstream, so we generated reporter constructs by fusing the genomic sequence from −400 to +30 of the start site of transcription to the firefly luciferase gene. In transfection experiments, the chimeric construct showed significant induction by either co-transfection with a constitutively active HIF proline mutant (P402A/P564G) or exposure of the transfected cells to 0.5% oxygen (FIG. 4d). Most noteworthy, when the reporter gene was transfected into the HIF-1α null cells, it did not show induction when the cells were cultured in hypoxia, but it did show induction when co-transfected with expression HIF-1α plasmid. We then generated deletions down to the first 36 bases upstream of transcription, and found that even this short sequence was responsive to HIF-1 (FIG. 4d). Analysis of this small fragment showed only one consensus HRE site located in an inverted orientation in the 5′ untranslated region. We synthesized and cloned a mutant promoter fragment in which the core element ACGTG was replaced with AAAAG, and this construct lost over 90% of its hypoxic induction. These experiments suggest that it is this HRE within the proximal 5′ UTR that HIF-1 uses to transactivate the endogenous PDK1 gene in response to hypoxia.

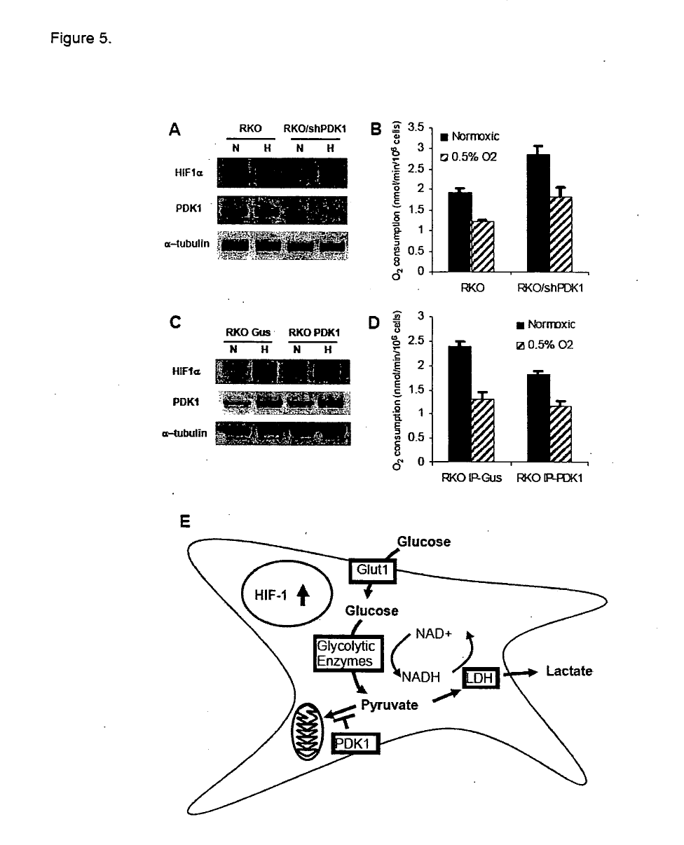

PDK1 is responsible for the HIF-dependent mitochondrial oxygen consumption changes. In order to directly test if PDK1 was the HIF-1 target gene responsible for the hypoxic reduction in mitochondrial oxygen consumption, we generated RKO cell lines with either knockdown or overexpression of PDK1, and measured the oxygen consumption in these derivatives. The PDK1 ShRNA stable knockdown line was generated as a pool of clones co-transfected with pSUPER ShPDK1 and pTK-hygro resistance gene. After selection for growth in hygromycin, the cells were tested by Western Blot for the level of PDK1 protein expression. We found that normoxic PDK1 is reduced by 75%, however, there was measurable expression of PDK1 in these cells in response to hypoxia (FIG. 5a). When we measured the corresponding oxygen consumption in these cells, we found a change commensurate with the level of PDK1. The knockdown cells show elevated baseline oxygen consumption and partial reduction in this activity in response to hypoxia. Therefore, reduction of PDK1 expression by genetic means increased mitochondrial oxygen consumption in both normoxic and hypoxic conditions. Interestingly, these cells still induced HIF-1α (FIG. 5a) and HIF-1 target genes such as BNip3L in response to hypoxia, suggesting that altered PDK1 levels do not alter HIF-1α function.

We also determined if overexpression of PDK1 could lead to reduced mitochondrial oxygen consumption. A separate culture of RKO cells was transfected with a PDK1-IRES-puro expression plasmid and selected for resistance to puromycin. The pool of puromycin resistant cells was tested for PDK1 expression by Western Blot. These cells showed a modest increase in PDK1 expression under control conditions when compared to the cells transfected with GUS-IRES-puro, with an additional increase in PDK1 protein in response to hypoxia (FIG. 5c). The corresponding oxygen consumption measurements showed that the mitochondria is very sensitive to changes in the levels of PDK1, as even this slight increase was able to significantly reduce oxygen consumption in the normoxic PDK1-puro cultures. Further increase in PDK1 levels with hypoxia further reduced oxygen consumption in both cultures (FIG. 5d). The model describing the relationship between hypoxia, HIF-1, PDK1, and intermediate metabolism is described in FIG. 5e.

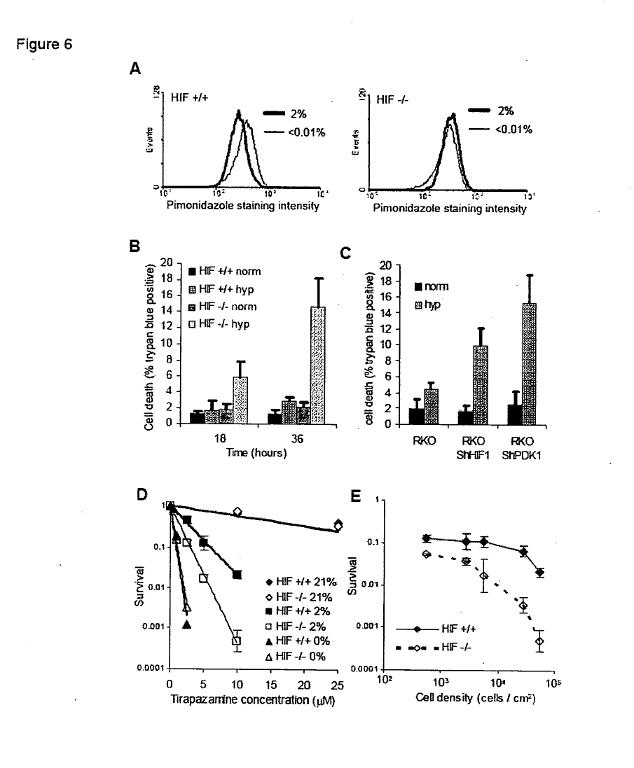

Altering oxygen consumption alters intracellular oxygen tension and sensitivity to hypoxia-dependent cell killing. The intracellular concentration of oxygen is a net result of the rate at which oxygen diffuses into the cell and the rate at which it is consumed. We hypothesized that the rate at which oxygen was consumed within the cell would significantly affect its steady state intracellular concentrations. We tested this hypothesis in vitro using the hypoxic marker drug pimonidazole (Bennewith and Durand, 2004). We plated high density cultures of HIF wild type and HIF knockout cells and placed these cultures in normoxic, 2% oxygen, and anoxic incubators for overnight treatment. The overnight treatment gives the cells time to adapt to the hypoxic conditions and establish altered oxygen consumption profiles. Pimonidozole was then added for the last 4 hours of the growth of the culture. Pimonidazole binding was detected after fixation of the cells using an FITC labeled anti-pimonidazole antibody and it was quantitated by flow cytometry. The quantity of the bound drug is a direct indication of the oxygen concentration within the cell (Bennewith and Durand, 2004). The histograms in FIG. 6a show that the HIF-1 knockout and wild-type cells show similar staining in the cells grown in 0% oxygen. However, the cells treated with 2% oxygen show the consequence of the genetic removal of HIF-1. The HIF-proficient cells showed relatively less pimonidazole binding at 2% when compared to the 0% culture, while the HIF-deficient cells showed identical binding between the cells at 2% and those at 0%. We interpret these results to mean that the HIF-deficient cells have greater oxygen consumption, and this has lowered the intracellular oxygenation from the ambient 2% to close to zero intracellularly. The HIF-proficient cells reduced their oxygen consumption rate so that the rate of diffusion into the cell is greater than the rate of consumption.

HIF-induced PDK1 can reduce the total amount of oxygen consumed per cell. The reduction in the amount of oxygen consumed could be significant if there is a finite amount of oxygen available, as would be the case in the hours following a blood vessel occlusion. The tissue that is fed by the vessel would benefit from being economical with the oxygen that is present. We experimentally modeled such an event using aluminum jigs that could be sealed with defined amounts of cells and oxygen present (Siim et al., 1996). We placed 10×106 wild type or HIF null cells in the sealed jig at 0.02% oxygen, waited for the cells to consume the remaining oxygen, and measured cell viability. We have previously shown that these two cell types are resistant to mild hypoxia, and equally sensitive to anoxia-induced apoptosis (Papandreou et al. 2005a). Therefore, any death in this experiment would be the result of the cells consuming the small amount of remaining oxygen, and dying in response to anoxia. We found that in sealed jigs, the wild type cells are more able to adapt to the limited oxygen supply by reducing consumption. The HIF null cells continued to consume oxygen, reached anoxic levels, and started to lose viability within 36 hours (FIG. 6b). We confirmed that it was PDK1 that was responsible for this difference by performing a similar experiment using the parental RKO cells, the RKOShRNAHIF1α and the RKOShRNAPDK1 cells. We found similar results in which both the cells with HIF1α knockdown and PDK1 knockdown were sensitive to the long term effects of being sealed in a jig with a defined amount of oxygen (FIG. 6c). Note that the RKOShPDK1 cells are even more sensitive than the RKOShHIF1α cells, presumably because they have higher basal oxygen consumption rates (FIG. 5b).

Because HIF-1 can help cells adapt to hypoxia and maintain some intracellular oxygen level, it may also protect tumor cells from killing by the hypoxic cytotoxin tirapazamine (TPZ). TPZ toxicity is very oxygen dependent, especially at oxygen levels between 1-4% (Koch, 1993). We therefore tested the relative sensitivity of the HIFwt and HIFko cells to TPZ killing in high density cultures (FIG. 6d). We exposed the cells to the indicated concentrations of drug and oxygen concentrations overnight. The cells were then harvested and replated to determine reproductive viability by colony formation. Both cell types were equally resistant to TPZ at 21% oxygen, while both cell types are equally sensitive to TPZ in anoxic conditions where intracellular oxygen levels are equivalent (FIG. 6a). The identical sensitivity of both cell types in anoxia indicates that both cell types are equally competent in repairing the TPZ-induced DNA damage that is presumed to be responsible for its toxicity. However, in 2% oxygen cultures, the HIF-null cells displayed a significantly greater sensitivity to the drug than the wild type cells. This suggests that the increased oxygen consumption rate in the HIF-deficient cells is sufficient to lower the intracellular oxygen concentration relative to that in the HIF-proficient cells. The lower oxygen level is significant enough to dramatically sensitize these cells to killing by TPZ.

If the increased sensitivity to TPZ in the HIFko cells is determined by intracellular oxygen consumption differences, then this effect should also be cell-density dependent. We showed that this is indeed the case in FIG. 6e where oxygen and TPZ concentrations were held constant, and increased cell density lead to increased TPZ toxicity. The effect was much more pronounced in the HIFko cells, although the HIFwt cells showed some increased toxicity in the highest density cultures, consistent with the fact they were still consuming some oxygen, even with HIF present (FIG. 1). The in vitro TPZ survival data is therefore consistent with our hypothesis that control of oxygen consumption can regulate intracellular oxygen concentration, and suggests that increased oxygen consumption could sensitize cells to hypoxia-dependent therapy.

Discussion.

The findings presented here show that HIF-1 is actively responsible for regulating energy production in hypoxic cells by an additional, previously unrecognized mechanism. It has been shown that HIF-1 induces the enzymes responsible for glycolysis when it was presumed that low oxygen did not support efficient oxidative phosphorylation. The use of glucose to generate ATP is capable of satisfying the energy requirements of a cell if glucose is in excess (Papandreou et al., 2005a). We now find that at the same time that glycolysis is increasing, mitochondrial respiration is decreasing. However, the decreased respiration is not because there is not enough oxygen present to act as a substrate for oxidative phosphorylation, but because the flow of pyruvate into the TCA cycle has been reduced by the activity of pyruvate dehydrogenase kinase.

This hypoxic shift in intermediate metabolism away from oxidative phosphorylation provides an elegant means for the cell to maintain both energy and redox states (diagrammed in FIG. 5). ATP is produced through the breakdown of glucose to pyruvate, but requires NAD as a cofactor for glyceraldehyde phosphate dehydrogenase (GAPDH), where it is reduced into NADH. NADH is routinely oxidized by the mitochondria to produce high energy electrons for electron chain transfer, and is regenerated to NAD for glycolysis. Under anoxic conditions, NADH is not recycled by the mitochondria, and so it must be regenerated by another means. The alternative non-oxygen requiring pathway is through the conversion of pyruvate to lactate by lactate dehydrogenase (LDH). Lactate is secreted into the extracellular space, and NAD is regenerated. The NAD cycle and redox state of the cell is therefore maintained through the activity of several interrelated systems, all of which are coordinately regulated by HIF-1 (glycolytic enzymes, pyruvate dehydrogenase kinase, and lactate dehydrogenase).

Inhibiting HIF-1 activity in the hypoxic tumor cells is predicted to have several therapeutic effects. One primary rationale for targeting HIF-1 is that it should only be activated within the hypoxic tumor microenvironment, so that therapy directed against HIF should not have systemic side effects. Current tumor models suggest that inhibiting HIF target genes such as VEGF will reduce tumor angiogenesis. One result of HIF inhibition is to increase oxygen consumption and make tumors more hypoxic. HIF inhibitors find use in clinical practice in conjunction with hypoxic cytotoxins, such as Tirapazamine.

### Example 2

Metabolic Targeting of Hypoxia and HIF1 in Solid Tumors can Enhance Cytotoxic Chemotherapy

Under hypoxic conditions, HIF1 causes an increase in its target gene PDK1, which acts to limit the amount of pyruvate entering the citric acid cycle, leading to decreased mitochondrial oxygen consumption. This adaptive response to low oxygen conditions may allow cells to spare molecular oxygen when it becomes scarce, making it available for other critical cellular processes. These findings predict that inhibition of HIF1 or PDK1 in vivo could alter tumor metabolism by increasing oxygen consumption which would lead to decreased overall tumor oxygenation. Decreased oxygenation in turn would increase the effectiveness of hypoxia targeted therapies such as the hypoxic cytotoxin tirapazamine. We tested this hypothesis using echinomycin, a recently identified small molecule inhibitor of HIF1 DNA binding activity, and dichloroacetate (DCA), a small molecule inhibitor of PDK1 activity.

Results

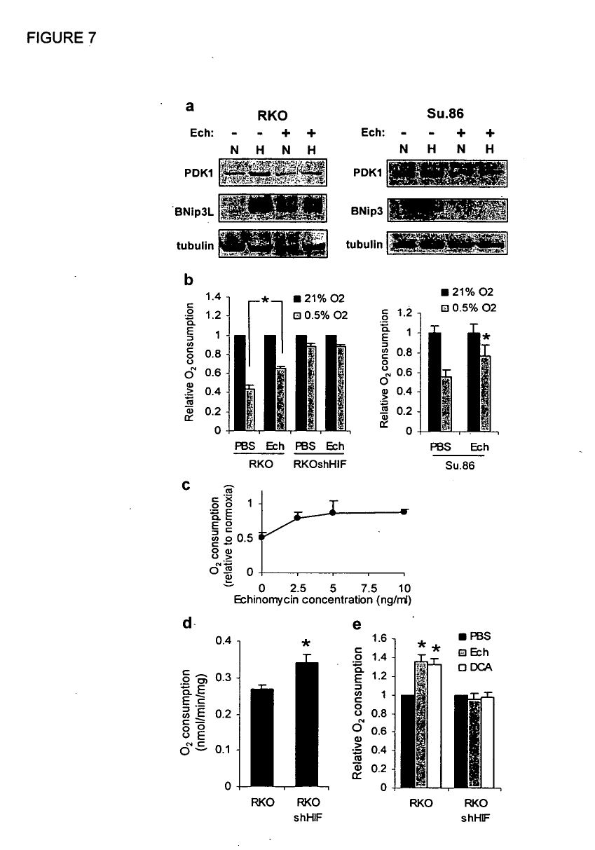

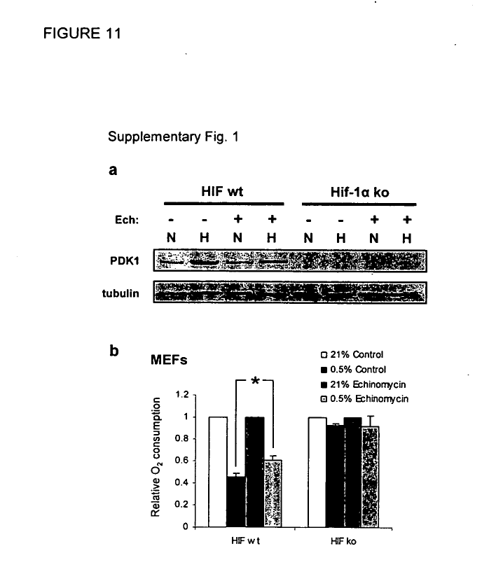

We first examined the effect of the HIF inhibitor echinomycin on the hypoxic expression of the HIF target gene PDK1 by immunoblot in RKO and Su.86 human tumor cells exposed to hypoxia. The HIF1 targets PDK1, Bnip3, and Bnip3L were induced by hypoxia, and this induction was blocked in the presence of echinomycin (FIG. 7a). To establish that this effect was due to HIF1 inhibition, we similarly tested wildtype and HIF1α knockout mouse embryo fibroblasts (MEFs). Echinomycin treatment blocked PDK1 induction in the wildtype cells, but had no effect on hypoxic expression of PDK1 in HIF1α deficient cells (FIG. 11a). Because PDK1 expression has been shown to inhibit oxygen consumption (17), and echinomycin blocks hypoxic PDK1expression, we tested echinomycin for its ability to modulate the hypoxic decrease in oxygen consumption. Echinomycin treatment yielded a dose-dependent block to the HIF1-dependent reduction in oxygen consumption in parental cells, and had no effect on the oxygen consumption in RKOShHIF1α cells (FIG. 7b,c). Similar effects of echinomycin on oxygen consumption were observed in the Su.86 (FIG. 7b) and MEF cell lines (FIG. 7b). These genetically matched cells show that echinomycin treatment can block the adaptive HIF1 dependent drop in oxygen consumption in wildtype cells, but has no effect on the oxygen consumption in either of the model HIF1 deficient cell lines (FIG. 7b,c).

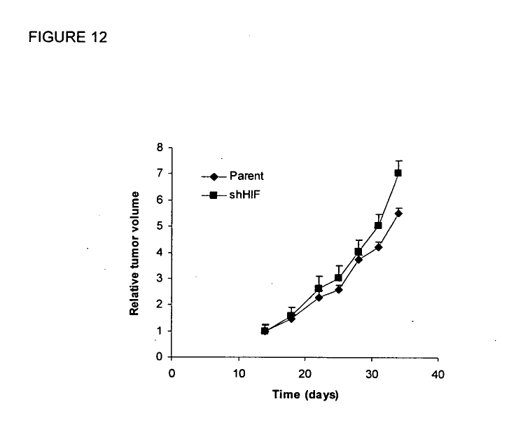

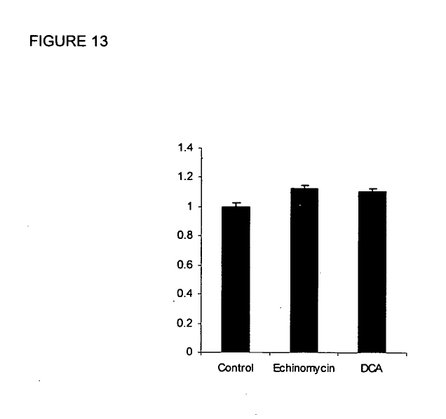

We next examined the effect of genetic and biochemical inhibition of HIF1 on oxygen consumption in vivo. Since HIF1 is not required for the growth of colon cancer xenografts (FIG. 12), RKO and RKOShHIF1α cells were grown as tumors in immune-deficient mice, and oxygen consumption was measured in freshly explanted samples. Oxygen consumption per milligram of tumor was significantly higher in HIF1 knockdown samples than in wildtype RKO samples (FIG. 7d). This is consistent with the existence of significant hypoxia in model tumors, and the in vitro finding that hypoxia decreases oxygen consumption in a HIF-dependant manner. To examine the effect of acute HIF1 inhibition in vivo, tumor bearing animals were treated with echinomycin and tumor oxygen consumption was measured. Echinomycin treatment significantly increased tumor oxygen consumption in RKO wildtype tumors but had no effect in RKOShHIF11 tumors, demonstrating that the pharmacologic target of echinomycin that can increase oxygen consumption in vivo is HIF1 (FIG. 7e). To test if this effect is mediated by the HIF1 target gene PDK1, we also measured oxygen consumption in tumors treated with the well-characterized PDK1 inhibitor DCA (21). Similar to the echinomycin treatment, DCA increased oxygen consumption in RKO tumors in a HIF1 dependent manner (FIG. 7e). These findings are consistent with our observation that alteration of PDK1 expression is able to influence oxygen consumption in vitro. Similar effects of both echinomycin and DCA were also observed in Su.86-derived tumors (FIG. 13).

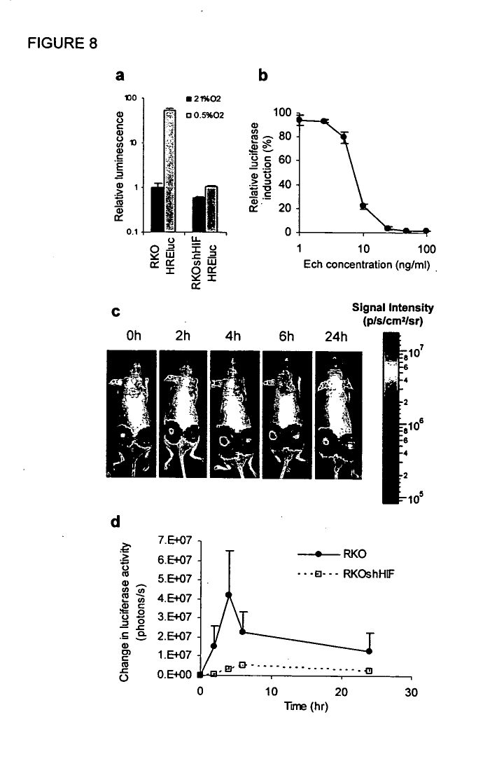

Tissue oxygen concentration is determined by both oxygen supply and oxygen demand. Mathematical modeling of tumor oxygenation suggests that small changes in oxygen consumption can have a large impact on the extent of tumor hypoxia when compared to changes in oxygen delivery. We therefore established a reporter system that would allow us to monitor the biologic changes in tumor oxygen levels in response to the observed changes in oxygen consumption caused by HIF1 or PDK1 inhibition. RKO and RKOShHIF11 cells were stably transfected with a luciferase reporter gene under the control of a synthetic HIF1 responsive promoter consisting of 5 tandem repeats of a HIF binding site (5×HRE). Luciferase activity in these cells provides a sensitive measure of hypoxia that can be monitored non-invasively over time both in vitro and in vivo using bioluminescent imaging. When RKO reporter cells were exposed to hypoxia for 24 hours in vitro, luciferase activity in wildtype cells increased approximately 80 fold, whereas the increase in the RKOShHIF1α cells was less than 2 fold (FIG. 8a). The hypoxic induction of luciferase in RKO reporter cells in vitro was completely inhibited by echinomycin in a dose dependent manner (FIG. 8b), consistent with the observed inhibitory effect of echinomycin on endogenous HIF1 target genes (FIG. 7a).

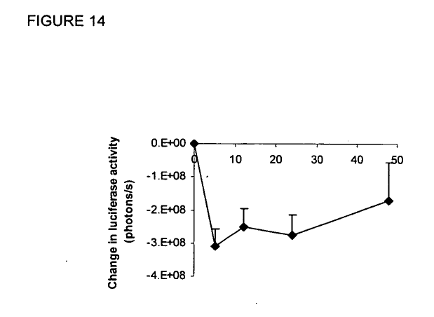

We next tested the effect of DCA treatment on luciferase activity in RKO and RKOShHIF1α 5×HRE-luciferase reporter tumors to determine if acutely increasing tumor oxygen consumption increases tumor hypoxia. Mice were implanted with one RKO and one RKOShHIF1α 5×HRE-luciferase reporter tumor on either flank, and luciferase activity was measured in vivo over time. After administration of DCA to the animal, the luciferase signal increased substantially in the wildtype, but not in the HIF1 knockdown tumors, supporting the hypothesis that increased oxygen consumption results in increased tumor hypoxia (FIG. 8b,c). To establish that the in vivo luciferase signal reflects the number of hypoxic tumor cells, a group of RKO reporter tumors was imaged over time following a single dose of the hypoxic cytotoxin tirapazamine. Twelve hours after treatment with tirapazamine, which has been shown to rapidly reduce the hypoxic fraction of experimental tumors by greater than 10-fold (28), the luciferase signal of HRE-reporter tumors was reduced by 90% (FIG. 14). Therefore, the luciferase signal emanating from the reporter tumors appears to be coming primarily from the hypoxic, tirapazamine-sensitive cells.

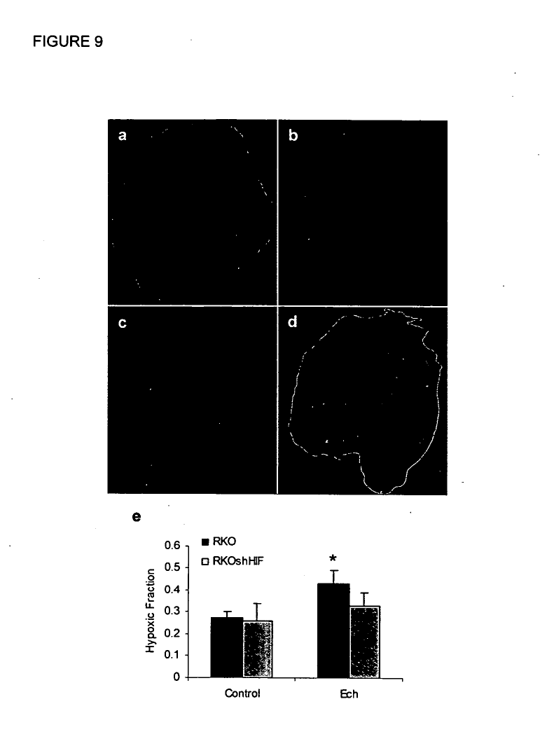

Because echinomycin acts as a HIF inhibitor, the 5×HRE reporter system could not be used to monitor the effect of the drug on tumor hypoxia. As an alternative, the hypoxia marker drug pimonidazole was used to determine the hypoxic fraction of tumors treated with echinomycin. Mice bearing RKO and RKOShHIF1α tumors were treated with echinomycin or vehicle control, and pimonidazole was administered at 24 h to identify the hypoxic tumor cells. FIG. 13 shows examples of sections from these tumors that have been stained with a FITC-conjugated monoclonal antibody against pimonidazole. The hypoxic fraction of each tumor was quantified by measuring the fraction of the viable tumor section that stained positive for pimonidazole. Echinomycin treatment caused an increase in the hypoxic fraction of RKO tumors, but had no significant effect on the extent of hypoxia in RKOShHIF1α tumors (FIG. 9e). There was no difference in other parameters such as the extent of necrosis observed in the four treatment groups.

Based on extensive experimental and clinical data, increasing the hypoxic fraction of solid tumors would be predicted to decrease the effectiveness of radiation therapy. However, hypoxic specific cytotoxins such as tirapazamine show increased toxicity as oxygen concentration decreases. Therefore, acutely increasing oxygen consumption and increasing the number of hypoxic tumor cells should enhance the efficacy of such drugs. To examine this possibility, we tested the ability of echinomycin and DCA to enhance the effectiveness of tirapazamine in a standard tumor growth delay assay. In both cases, treatment of RKO tumor bearing animals with the metabolic modifier prior to tirapazamine produced greater than additive tumor growth delay compared to single agents (FIG. 9a,b). If echinomycin is acting as a metabolic modifier to enhance tirapazamine therapy, it should only be effective if given before the hypoxic cytotoxin. We tested this prediction by reversing the drug schedule, and found that treatment with tirapazamine followed by echinomycin had little effect on the growth of RKO or Su.86 tumors when compared to treatment with echinomycin followed by tirapazamine (FIG. 9c,e). To determine if the molecular target of echinomycin in vivo responsible for its ability to sensitize tumors to tirapazamine was indeed HIF1, we performed a similar growth delay experiment using RKOShHIF11 tumors. In this situation, echinomycin plus tirapazamine had no significant effect on tumor growth, regardless of the order that the drugs were given (FIG. 9d). This data provides genetic evidence that biochemical inhibition of HIF1 or its target gene PDKL alters the metabolism of the tumor, increases the degree of hypoxia, and sensitizes tumors to treatment with hypoxia specific cytotoxins such as tirapazamine.

Discussion

HIF1 has been shown to decrease mitochondrial oxygen consumption through the upregulation of its target gene PDK1, suggesting that inhibition of this pathway may represent a novel means of specifically altering the hypoxic microenvironment of solid tumors. The model predicts that inhibiting the transcriptional activity of HIF in solid tumors will block the HIF-mediated adaptive response to hypoxia and result in an increase in the rate of oxygen consumption. This increased consumption of oxygen should lead to an increase in the extent of tumor hypoxia. Since hypoxia and HIF stabilization are situations encountered primarily in solid tumors, this strategy should not affect the metabolism of normal tissues.

Here we show that biochemical inhibition of HIF or PDK1, by echinomycin or DCA respectively, increases the oxygen consumption rate of solid tumors. As predicted, this intervention results in an increase in tumor hypoxia as measured using a bioluminescent hypoxia reporter system, or the hypoxia marker drug pimonidazole. Importantly, unlike other methods of measuring tumor hypoxia, both of these techniques rely on the presence of viable hypoxic cells. These findings are consistent with models suggesting that tumor oxygenation should be very sensitive to changes in oxygen consumption rates. Interestingly, although genetic inhibition of HIF1α in RKO cells resulted in increased oxygen consumption in the RKOShHIF1α tumors, the hypoxic fraction and necrotic fraction of these tumors were not different from those of control tumors. This suggests that during the course of tumor development, oxygen delivery may be increased in the HIF1α knockdown tumors, compensating for the increased oxygen demand.

Hypoxia decreases the effectiveness of radiation therapy and certain types of traditional chemotherapy, and predicts for poor outcome in several human malignancies. Further, it has been shown to accelerate tumor progression and metastasis in experimental system. Unfortunately, attempts to improve tumor oxygenation during therapy have not yielded clinically compelling results. The alternative is to exploit the hypoxic microenvironment by designing therapies that take advantage of this unique property of solid tumors. Approaches currently under investigation include the use of anaerobic bacteria, hypoxia-specific gene therapy vectors, and bioreductive drugs that are converted to their cytotoxic forms under low oxygen conditions. Contrary to conventional treatments, it is predicted that these therapies should be more effective against more hypoxic tumors. In the case of tirapazamine, recent experimental and clinical data support this concept, as the drug was found to have greater efficacy in treatment of more hypoxic tumor xenografts (Emmenegger et al. (2006) Cancer research 66, 1664-74), and patients with more hypoxic tumors (Rischin et al. (2006) Journal of clinical oncology 24, 2098-104). We show here that decreasing tumor oxygenation by increasing oxygen consumption sensitizes tumors to treatment with tirapazamine. The lack of an effect in the RKOShHIF1α tumors demonstrates that the increased sensitivity is due to echinomycin's interaction with HIF. Furthermore, the requirement that echinomycin be given prior to tirapazamine in order to achieve any increase in tumor growth delay shows that it is not acting by directly killing a complementary population of tumor cells, but rather by altering the tumor microenvironment. This novel approach to the modification of tumor hypoxia should prove useful for other bioreductive drugs, and also for other treatment strategies that rely on the presence of tumor hypoxia. This finding also suggests that targeting HIF may decrease the effectiveness of radiation therapy, due to the increase in radiobiologically hypoxic tumor cells. More generally, these data emphasize that care should be taken in designing and scheduling therapeutic regimens that include agents capable of modifying the tumor microenvironment in order to ensure that other cytotoxic components of the treatment are given at the optimal time.

Currently, there is a great deal of interest in developing specific and potent HIF inhibitors for a variety of clinical applications (Melillo (2006) Molecular cancer research 4, 601-5). Specifically, it has been suggested that HIF inhibitors may possess anti-tumor activity based on their effect on the tumor vasculature (Hickey & Simon=(2006) Current topics in developmental biology 76, 217-57; Kung et al. (2004) Cancer cell 6, 33-43). The results reported here provide a novel mechanism of action by which these new inhibitors may be used therapeutically.

Materials and Methods

Cell lines and tumor xenografts. RKO human colon carcinoma cells and Su.86 human pancreatic carcinoma cells were obtained from the American Type Culture Collection (ATCC, Manassas, Va.). The RKOShHIF1α cell line in which HIF1α is stably knocked down by shRNA has been described previously. RKO and immortalized MEF cells were grown in Dulbecco's Modified Eagle's Media (DMEM) supplemented with 10% fetal bovine serum (FBS). Su.86 cells were grown in RPMI 1640 medium supplemented with 10% FBS. Cells were exposed to hypoxia by placing culture dishes into an Invivo₂ humidified hypoxia workstation (Ruskin Technologies, Bridgend, UK) at 0.5% O2. Echinomycin (a gift of A. Giaccia, Stanford University) was included in the media at the indicated concentrations. Tumor xenografts were established by injecting 5×10⁶ cells for the RKO lines or 1×10⁷ cells for the Su.86 line sc into the flanks of 6-8 week old female nude mice. To monitor tumor growth, caliper measurements were made of two perpendicular diameters, and the formula (d₁)(d₂)(d₂)(0.52) was used to calculate tumor volume. All animal protocols were approved by the Stanford Administrative Panel on Laboratory Animal Care.

Western blots. In brief, cells were harvested directly in RIPA buffer containing protease inhibitors, protein concentrations were quantified (Pierce), 25-50 μg of total protein was electrophoresed on a reducing Tris-Tricine gel, and electroblotted to PVDF membrane. Antibodies used were rabbit anti-PDK1 (Stressgen) (1:2000), murine anti-α tubulin (Research Diagnostics) (1:2000), and rabbit anti-Bnip3 and anti-Bnip3L as described previously by Papandreou et al. (2005) Cancer research 65, 3171-8. (1:500). Primary antibodies were detected with species-specific secondary antibodies labeled with Alkaline Phosphatase (Vector labs) (1:3000) and visualized with ECF (Amersham) on a Storm 860 phosphoimager (Molecular Devices).

Oxygen consumption measurements. Cells were trypsinized and suspended at 3×10⁶ to 6×10⁶ cells per ml in normoxic DMEM+10% FBS. Oxygen consumption was measured in a 0.5 ml volume using an Oxytherm electrode unit (Hansatech, Norfolk, UK). This system employs a Clark-type oxygen electrode to monitor the dissolved oxygen concentration in a sealed measurement chamber over time. The data are exported to a computerized chart recorder (Oxygraph 1.01, Hansatech, Norfolk, UK), which calculates the rate of oxygen consumption. A small stir bar maintains the cells in suspension, and a Peltier heating block maintains the temperature at 37° C. Background electrode consumption was subtracted from each measurement. To measure in vivo oxygen consumption, tumors were excised and four samples per tumor, of approximately 50 mg each, were weighed and thoroughly minced in DMEM+10% FBS. Oxygen consumption was measured as above in a 1 ml volume and normalized to tissue weight.

Luciferase reporter assay. RKO and RKOshHIF1α cells were stably transfected with a luciferase reporter construct (5×HRE-luciferase) containing the firefly luciferase gene under the control of a synthetic HIF responsive promoter described previously by Shibata et al. (2000) Gene therapy 7, 493-8. Cells were exposed to 0.5% O2 for 24 hr, and luciferase activity was measured in triplicate using a luciferase reporter gene assay kit (Roche) and a Monolight 2010 luminometer (Analytical Luminescence Laboratory). For analysis of the effect of echinomycin on luciferase activity in vitro, 5×10⁴ cells were seeded to 96 well plates and 24 hours later, media was changed and drugs added. Plates were placed in normoxic or hypoxic (0.5% O2) incubators for 24 hours and imaged directly in a Xenogen IVIS100 bioluminescent imaging system (Xenogen, Alameda, Calif.) in the presence of 150 μg/ml potassium d-luciferin (Xenogen).

Bioluminescent imaging. Mice bearing 100-200 mm³ subcutaneous HRE-luciferase reporter tumors were anesthetized using 2% isofluorane and injected ip with 150 mg/kg potassium d-luciferin (Xenogen). After 10 min, bioluminescence was measured in a Xenogen IVIS100 imaging system (Xenogen). Data was quantified by measuring total photons/s from uniform regions of interest. The data are presented as the change in bioluminescence relative to pretreatment values. Data points represent the mean of 3 independent experiments, each comprising 5 RKO and 5 RKOShHIF1 tumors.

Detection of tumor hypoxia by pimonidazole immunofluorescence. Mice bearing 100-300 mm³ subcutaneous RKO and RKOshHIF tumors on either flank were treated with 0.12 mg/kg echinomycin ip or saline control, and 24 h later they were injected with the hypoxia marker drug pimonidazole (60 mg/kg ip) (Millipore, Temecula, Calif.). Three hours after pimonidazole injection, tumors were excised, and frozen in Tissue-Tek O.C.T. compound. Frozen sections (10 μm thick) from the center regions of tumors were cut, air dried, and fixed for 15 min in acetone at 4 C. Slides were then air dried, rehydrated in PBS, and blocked for 30 min in 4% FBS, 5% non-fat milk, and 0.1% Triton X-100 in PBS. Slides were incubated for 1 hr at room temperature with a FITC-conjugated monoclonal antibody against pimonidazole (Millipore) diluted 1:20 in blocking solution. Slides were washed, counterstained with 50 nM propidium iodide (PI), and mounted under a coverslip in Vectashield medium (Vector Labs, Burlingame, Calif.).

FITC and Pi fluorescent signals for entire tumor sections (one section per tumor) were acquired using a 4× objective lens on a Nikon Eclipse E800 microscope equipped with a motorized scanning stage, a 12-bit QImaging camera (OImaging, Burnaby, Canada), and Bioquant imaging software (Bioquant, Nashville, Tenn.). Acquisition parameters were held constant for all samples. ImageJ software (NIH, Bethesda, Md.) was used to analyze the resulting tiled images. The area of the tumor section was manually defined using the PI signal, and large areas of necrosis and cutting artifacts were removed. The FITC positive area was then defined using a common threshold value for all tumor sections. The threshold value was chosen such that all signal was eliminated on control tumor sections from animals not injected with pimonidazole. The hypoxic fraction was defined as the FITC positive area/the viable tumor area.

Tumorgrowth delay. Female nude mice were implanted sc with RKO, RKOShHIF1α, or Su.86 tumors as described above. When the mean tumor volume reached 100-200 mm³, mice were randomized to treatment groups. The echinomycin plus tirapazamine groups were treated with 0.12 mg/kg echinomycin ip followed at 24 h by 30 mg/kg tirapazamine ip, followed by a rest day for 3 or 6 cycles as indicated. The DCA plus echinomycin treatment group was treated with 50 mg/kg DCA ip followed at 4 hours by 20 mg/kg tirapazamine ip daily for 14 days. Single agents were given at the same doses on the same schedules. Control animals were given ip injections of saline on the same schedule. For schedule dependence experiments, the doses were the same as above, with the tirapazamine plus echinomycin group receiving tirapazamine, followed at 24 hours by echinomycin, followed by a rest day for 6 cycles. Each treatment arm consisted of 2 independent groups of 6-8 tumors (12-16 total tumors per group).

Data Analysis. Changes in oxygen consumption, luciferase activity, hypoxic fraction, and tumor growth were analyzed by ANOVA, followed by pair-wise comparisons using a two-tailed Student's t-test with the Bonferroni correction for multiple comparisons as needed. For all data, p-values<0.05 were considered significant. All error bars represent the standard error of the mean.

What is claimed is:

1. A method for treatment of cancer, the method comprising:
contacting a targeted cancer cell population with a combination of an inhibitor of HIF-1 and/or PDK; and a hypoxic cytotoxin;
in a combined dosage effective to substantially reduce the numbers of said targeted cancer cell population.

2. The method of claim 1, wherein said cancer is a human cancer.

3. The method of claim 2, wherein said cancer is a solid tumor.

4. The method of claim 3, wherein said cancer is a carcinoma.

5. The method of claim 1, wherein said hypoxic cytotoxin is converted to a cytotoxic form under hypoxic conditions.

6. The method of claim 5, wherein said hypoxic cytotoxin is chosen from quinones based on the indolequinone nucleus; nitroheterocyclic compounds; aromatic N-oxides; and aliphatic N-oxides.

7. The method of claim 6, wherein said hypoxic cytotoxin is tirapazamine.

8. The method of claim 1, wherein said inhibitor is a HIF-1 inhibitor.

9. The method of claim 1, wherein said inhibitor is a PDK inhibitor.

10. The method of claim 1, wherein said inhibitor of HIF-1 and/or PDK; and hypoxic cytotoxin are administered in a co-formulation.

11. The method of claim 1, wherein said inhibitor of HIF-1 and/or PDK; and a hypoxic cytotoxin are separately formulated.

12. The method according to claim 1, wherein said combination of inhibitor of HIF-1 and/or PDK; and a hypoxic cytotoxin provide for a synergistic response.


SEQUENCE LISTING (2 sequences)

SEQ ID NO: 1 - RNA, 19 nt, human
ugaggaaguaccauuauau

SEQ ID NO: 2 - RNA, 25 nt, human
cgacacaaugaugucauucccacaa